SEQUENCE LISTING (9 sequences)

SEQ ID NO: 1 - protein, 199 aa, synthetic construct
RPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 2 - protein, 239 aa, synthetic construct
MVSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 3 - protein, 208 aa, synthetic construct
RELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPII

SEQ ID NO: 4 - protein, 168 aa, synthetic construct
QEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTA

SEQ ID NO: 5 - protein, 586 aa, synthetic construct
MSSSFLSSTAFFLLLCLGFCHVSSLLPKLERSAARPSGEPGCSCAQPAAEAAAPGWAQARGHPGGELEAAASAAGDAGWPNKHTLRILQDFSSDPSSNLTSHSLEKLPPAAEAAEGAPPGQDPGVRRPPDPAHRPLPRDPGPRGPVLPPGLSGDGSLLTRLFQHPLYQVPIPPLTEGDVLFNVNSDIRFNPKAATAENPDWPHEGPEDEFLPTGEAAVDSYPNWLKFHIGINRYELYSRHNPAVGALLQDLGTQKITSVAMKSGGTQLKLIMTFQNYGQALFKPMKQTREQETPPDFFYFSDYERHNAEIAAFHLDRILDFRRVPPVAGRLVNMTKEIRDVTRDKKLWRTFFISPANNVCFYGECSYYCSTEHALCGKPDQIEGSLAAFLPDLALAKRKTWRNPWRRSYHKRKKAEWEVDPDYCEEVRQTPPYDSSHRLLDVMDMTIFDFLMGNMDRHHYETFEKFGNETFIIHLDNGRGFGKHSHDELSILVPLQQCCRIRRSTYLRLQLLAQEEHRLSLLMAEALRADRVAPVLFQPHLEALDRRLRIVLRAVGDCVEKDGLHSVVEDDLGPEHRAAAGRHDEL

SEQ ID NO: 6 - DNA, 60 bp, synthetic construct
  source 1..60: mol_type = other DNA
agttcggattacaaagatgacgacgataagcatcatcaccatcaccaccatgatgagttg

SEQ ID NO: 7 - DNA, 57 bp, synthetic construct
  source 1..57: mol_type = other DNA
tcggattacaaagatgacgacgataagcatcatcaccatcaccaccatgatgagttg

SEQ ID NO: 8 - protein, 19 aa, synthetic construct
SDYKDDDDKHHHHHHHDEL

SEQ ID NO: 9 - DNA, 12902 bp, synthetic construct
  source 1..12902: mol_type = other DNA
gtgccgaattcggatccggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagttaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgccatggccaagctagttttttccctttgttttctgcttttcagtggctgctgcttcgcaatgcgtccgaagcacccaatcaagcatcaagggcttccccaagaggtactgaacgaaaatctactcagattcttcgtggctccatttcctgaggtatttgggaaggaaaaggtgaatgagctgtctaaagacataggctctgaatcaactgaggaccaagctatggaggatataaagcaaatggaggccgaatccatatcctcaagcgaggaaatcgttccgaattccgtcgaacagaagcatattcaaaaggaggatgtgccttctgagcgataccttggatatcttgaacagttgctgagactaaagaaatacaaggtcccccaactagaaattgttcctaactcagccgaagagcgattacactctatgaaagaggggattcatgctcaacagaaggagcctatgatcggtgtgaatcaagaactggcatacttctatcccgaactgtttcgacaattttatcaattagatgcgtatccatccggggcgtggtattatgtgcctcttgggacacagtatacggatgcgccgagtttttcagacatcccaaacccaataggcagtgagaattcagaaaagacgacaatgcctctttggagttcggattacaaagatgacgacgataagcatcatcaccatcaccaccatgatgagttgtaggcttatatgtcaacagtgagaaactgttcgcattttccgttttgcttctttctttctattcaatgtatgttgttggattccagttgaatttattatgagaactaataataatagtaataatcatttgtttctttactaatttgcattttcacatatgatttctggtgcatatcataattttcattccaccaatattaatttcccccattcaagttacttatgaaatagaaatcctcttctccgactactttatttgtccgaaagtcttgtggctgctatataacgctgcaagaattcaagcttagcggtcctgctgagcctcgacatgttgtcgcaaaattcgccctggacccgcccaacgatttgtcgtcactgtcaaggtttgacctgcacttcatttggggcccacatacaccaaaaaaatgctgcataattctcggggcagcaagtcggttacccggccgccgtgctggaccgggttgaatggtgcccgtaactttcggtagagcggacggccaatactcaacttcaaggaatctcacccatgcgcgccggcggggaaccggagttcccttcagtgagcgttattagttcgccgctcggtgtgtcgtagatactagcccctggggcacttttgaaatttgaataagatttatgtaatcagtcttttaggtttgaccggttctgccgctttttttaaaattggatttgtaataataaaacgcaattgtttgttattgtggcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcttctcgcatatctcattaaagcaggacaagcttacttgtacagctcgtccatgccgtgagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtagctgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccattgtatcgataattgtaaatgtaattgtaatgttgtttgttgtttgttgttgttggtaattgttgtaaaaatgagctcttatactcgagcgtgtcctctccaaatgaaatgaacttccttatatagaggaagggtcttgcgaaggatagtgggattgtgcgtcatcccttacgtcagtggagatgtcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttgacctccactagaattcgagctcggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagttaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgccatggccaagctagttttttccctttgttttctgcttttcagtggctgctgcttcgcaatgagagaacttgaagaattgaatgttccaggagagattgttgaaagtctctcctcttctgaggaaagcatcacaagaatcaacaagaaaattgagaagtttcagagtgaagagcaacaacagactgaagatgaattacaagataagattcatccttttgctcaaacacagtcacttgtgtatccattccctggaccaattccaaattctttaccacaaaacattcctcctctgactcaaactcctgtcgttgttcctccgttcttgcaaccagaagttatgggagtttcaaaggttaaagaagcaatggctccaaagcataaagagatgccattcccaaaataccctgtggagcctttcacagaatctcaaagcttgactctcactgatgttgagaatcttcatttgcctcttccattgcttcaatcatggatgcatcaacctcatcagcctttgccaccaacagtgatgtttccacctcaatctgttctctctctttctcagtctaaagttcttccggttccgcagaaagctgtgccttatcctcagagagatatgcctattcaagcttttcttctctaccaagaaccagttttgggtcctgttcgtggtccatttcccatcatagtttcggattacaaagatgacgacgataagcatcatcaccatcaccaccatgatgagttgtaggcttggactcccatgttggcaaaggcaaccaaacaaacaatgaatgatccgctcctgcatatggggcggtttgagtatttcaactgccatttgggctgaattgtagacatgctcctgtcagaaattccgtgatcttactcaatattcagtaatctcggccaatatcctaaatgtgcgtggctttatctgtctttgtattgtttcatcaattcatgtaacgtttgcttttcttatgaattttcaaataaattatccgctttacgaattcccatggagcgctattactcaaattctcattaatttcaagaattttcttaatttcacagtatacaatttaaattcagaagaattgcaataggatgaatcaattaagaaaaattaatcaacagctaaaagacgaaccaatgtcatcactagaaactgatatctattcattgatactgtaatgagaaattacaagatctaagccataagaacagcactacagatatccaacacagacatacgcaattgtaaactaaatccctacatttaccaccatatcaccaacataaaagcctacaactcatcatggtggtgatggtgatgatgcttatcgtcgtcatctttgtaatccgaaacagccgttgaggtgacctgtaccgtgtttatctccgggggtgattcaatcacctctggggaatcttcaagtgtggcgaccgtgctctccaccgcttcagtagtcggtgttgaagttggctcaccagaagcgatagtatttatggtcgggatctctgtcttgtcttggttctttttgggaggaatagccataaaggaaaggtgcggatgcgggtgtctagccatggtggtgggttgcgcctggcaagacttcgccgggacagtgttggataaaacctgccactgcaatatctgagcaggagaacggacggcggccggcttagcatagtagggatatgggagaaactggttgtttatcaaggcaactggcttctgttgatagtaattaagaccgtagctcggatatcgacttaaaacgtactggattgggatgtactttgcaattttatcactaaaaaatctctcgtctttttcacaacgaataggttgctcttggttttgctcctgcattgcgaagcagcagccactgaaaagcagaaaacaaagggaaaaaactagcttggccatggcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttaactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcccagagaattcgcatgcggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatgagcagcagtttcttgagctcaaccgctttctttctattactctgtctggggttctgtcacgtgagcagtctcctacccaaattagaacgatctgctgcacgtccgagcggcgaacctggctgtagctgcgcacagccagctgccgaagctgccgcgcctggatgggctcaagctaggggtcatcccggtggagaacttgaagcggccgctagcgccgccggggatgcaggctggccaaataagcacactctgaggattctgcaagacttcagttccgaccccagttccaacctaacgagccactcactggaaaagctgcctccggctgccgaagctgcggaaggtgcaccgccaggccaagatccaggagttcgtagacctcccgacccagcgcataggccactcccgcgagatccgggtcctagaggccctgtcttgcccccaggtcttagcggagacgggtccttacttacgcgtcttttccaacacccgctataccaggtgcccataccgcccctaacagaaggcgatgttctctttaatgtcaatagcgatataagattcaaccccaaagctgcaaccgccgagaacccagattggccacacgaggggccggaagatgagtttttacctactggtgaagcggcagttgactcttacccgaattggctgaagtttcatattgggatcaatagatacgagctttacagccgacataatccggccgtgggagcgctcttacaagacctcgggacgcaaaagattacttctgtcgctatgaaatctggcgggacacagctcaaacttattatgactttccagaattatggccaagctctgttcaagccgatgaagcagactagagagcaggagacaccccctgacttcttctacttcagcgactatgaaaggcataatgcagaaattgcggcattccaccttgataggatcttagacttccgaagggtaccaccggtagcaggtagactagtcaatatgactaaagagattagagatgtcactcgtgacaagaaactatggcgtacattctttataagccctgctaacaatgtatgcttttatggcgaatgttcttactattgctctacagaacatgcactgtgtggaaaacccgaccagattgaggggtcactagccgcatttctgccagacttggcattggccaagcgtaagacgtggcgtaatccgtggcgacgtagttaccacaagagaaagaaggcggagtgggaagtagacccagactactgcgaggaggttagacaaacacctccatatgattctagtcatagactgttggatgttatggacatgacaatttttgattttctcatggggaacatggatcgtcaccactacgaaacctttgagaaattcggcaatgagacattcattatccacttagataatggtcgaggttttggcaaacacagccatgacgaactatctatattagtgcctttacaacagtgctgtagaatccgaaggtctacctatttgagacttcaactgttggcccaagaggagcatcgtctatcacttttaatggccgaggctctaagggctgatcgtgtggctcccgtactctttcagcctcacttagaggctttagatcgtcgacttcgtatagtgcttcgagcggtaggcgattgcgtggagaaagatggactgcacagtgttgtagaggatgatttggggcctgagcacagggcggccgcgggacgtcacgatgagttataagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgcttgtggaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

(19) United States
(12) Patent Application Publication
(10) Pub. No.: US 2025/0311748 A1
(43) Pub. Date: Oct. 9, 2025

(54) MODIFIED CASEIN PROTEINS

(71) Applicant: MOZZA FOODS, INC., Los Angeles, CA (US)

(72) Inventors: Cory J. Tobin, Los Angeles, CA (US); Noah Brookes, Los Angeles, CA (US)

(73) Assignee: MOZZA FOODS, INC., Los Angeles, CA (US)

(21) Appl. No.: 18/855,214

(22) PCT Filed: Apr. 7, 2023
(86) PCT No.: PCT/US2023/065558

§ 371 Date: Oct. 8, 2024

Related U.S. Application Data
(60) Provisional application No. 63/329,358, filed on Apr. 8, 2022.

(51) Int. Cl. A23J 3/10; A01H 6/54; A23J 1/00; C12N 1/14; C12N 1/20; C12N 15/11; C12N 15/867

(52) U.S. Cl. CPC A23J 3/10; A01H 6/542; A23J 1/006; C12N 1/14; C12N 1/20; C12N 15/11; C12N 15/867; C12N 2740/10043

(57) ABSTRACT

Compositions, methods and systems for producing functional non-glycosylated, under-glycosylated, or differentially-glycosylated recombinant casein proteins in plants which allow for the safe, sustainable, and humane production of milk proteins for commercial use, such as in food compositions. The disclosure provides modified non-glycosylated, under-glycosylated, or differentially-glycosylated recombinant casein protein, or fragments thereof. The disclosure also provides methods for producing modified non-glycosylated, under-glycosylated, or differentially glycosylated recombinant proteins, and food compositions comprising the same.

### CROSS-REFERENCE

This application claims the benefit of International Patent Application Serial No. PCT/US2023/065558, filed on Apr. 7, 2023, which claims the benefit of U.S. Provisional Application No. 63/329,358, filed on Apr. 8, 2022, which is incorporated herein by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SEQUENCE LISTING

The instant application contains a Sequence Listing which has been submitted electronically in XML format and is hereby incorporated by reference in its entirety. Said XML copy, created on Oct. 7, 2024, is named 705601002_SEQLIST.xml and is 23 kilobytes in size.

### BACKGROUND

Glycosylation is the reaction in which a carbohydrate (or “glycan”) is attached to a hydroxyl or other functional group of another molecule (a glycosyl acceptor) in order to form a glycoconjugate. Glycosylation is a form of enzyme-catalyzed co-translational and posttranslational modification that occurs in most cells with the majority of proteins synthesized in the rough endoplasmic reticulum undergoing glycosylation.

Casein micelles are important for milk and cheese production. Casein micelles are made of one or more of alpha-casein, beta-casein, and kappa-casein proteins. Kappa-caseins made in mammals are glycosylated by glycans which are negatively charged and hydrophilic. These glycans impact kappa-casein interactions with other molecules in beneficial ways as they enhance micelle formation and function, solubility of casein proteins and casein micelles, milk production, and curd formation.

Since glycosylation pathways are not conserved across species, recombinant proteins that are glycosylated in their native host, are frequently not glycosylated or under-glycosylated when produced in non-native host organisms. Recombinant proteins which are not glycosylated by their host cell, or those that have lower levels of glycosylation than native forms, may be less stable, less soluble, or fold improperly. These issues can negatively impact recombinant protein function and production levels in host systems as well as limit the formation of higher level functional structures.

Making proteins glycosylated by mammalian glycans in plants is difficult, because the native glycosylation pathways in plants produce far fewer glycans than in mammalian cells, with only two glycoforms accounting for >90% of glycans in plant proteins. Further, plant glycans are richer in xylose and fucose residues and also contain unique Lewis A structures not found in mammalian glycoproteins. Since glycosylation pathways are not conserved across species, recombinant proteins that are glycosylated in their native host, are frequently not glycosylated or under-glycosylated when produced in non-native host organisms. Recombinant proteins which are not glycosylated by their host cell, or those that have lower levels of glycosylation than native forms, may be less stable, less soluble, or fold improperly. These issues can negatively impact recombinant protein function and production levels in host systems as well as limit the formation of functional higher level structures.

### SUMMARY

The current disclosure provides compositions, methods and systems in which recombinant casein proteins are modified to achieve the same or similar biochemical properties as provided by glycosylation by mammalian glycans, when the recombinant casein proteins are expressed in non-mammalian or non-animal organisms. In some cases, the modified casein proteins are non-glycosylated, under-glycosylated, or differentially glycosylated but still possess the same or similar biochemical properties as provided by glycosylation by mammalian glycans. In some cases, the modified casein proteins are glycosylated in a plant by plant glycans (e.g., high levels in xylose and fucose residues, with unique Lewis A structures), but still possess the same or similar biochemical properties as provided by glycosylation by mammalian glycans.

In some cases, the mammalian glycan-related protein functionality comprises increased solubility of the mutant casein protein in a liquid. In some cases, the mammalian glycan-related protein functionality comprises increased stability of the mutant casein protein in a liquid. In some cases, the mammalian glycan-related protein functionality comprises enhanced casein micelle formation. In some cases, the mammalian glycan-related protein functionality comprises improved milk production. In some cases, the mammalian glycan-related protein functionality comprises improved curd formation in a cheese making process. In some instances, the mammalian glycan-related protein functionality comprises an improved dairy characteristic.

In some aspects, the disclosed compositions, methods and systems allow for the production of casein proteins, and in particular, functional casein proteins and casein micelles with desirable characteristics in non-animal organisms. In some cases, the non-glycosylated, partially-glycosylated, or differentially glycosylated proteins are produced in transgenic plants. In some cases, the non-glycosylated, partially-glycosylated proteins or differently-glycosylated caseins are produced in yeast, fungus, and bacteria.

In some aspects, the present disclosure relates to recombinant proteins that are non-glycosylated, under-glycosylated, or differentially glycosylated and methods of improving expression, solubility, stability, and functionality of these proteins in transgenic host organisms. In some aspects, the present disclosure also relates to food compositions comprising recombinant proteins that are non-glycosylated, under-glycosylated, or differentially glycosylated.

In some aspects, the current disclosure provides compositions, methods and systems for improving the functional characteristics of non-glycosylated, under-glycosylated or differentially glycosylated proteins in plants. In some cases, the current disclosure provides vectors for expressing proteins in a plant where hydrophobic residues are replaced with hydrophilic residues providing mammalian glycan-related protein functionality, for example, micelle formation and stability, in the absence of mammalian glycans.

In some aspects, the current disclosure provides a mutant casein protein comprising at least one mutation compared with a wild-type casein protein counterpart, wherein the at least one mutation provides mammalian glycan-related protein functionality. In some cases, the mutant casein protein is a mutated αS1-casein, αS2-casein, ß-casein, or κ-casein. In some cases, the at least one mutation is located on the caseinomacropeptide of the mutated κ-casein. In some cases, the at least one mutation is located on the N-terminal of the caseinomacropeptide of the mutated κ-casein. In some cases, the at least one mutation is located at C-terminal caseinomacropeptide of the mutated κ-casein.

In some cases, the at least one mutation comprises changing at least one hydrophobic amino acid to a hydrophilic amino acid. In some cases, the at least one mutation comprises changing at least two hydrophobic amino acids to hydrophilic amino acids. In some cases, the at least one mutation comprises changing at least three hydrophobic amino acids to hydrophilic amino acids. In some cases, the at least one mutation comprises changing at least six hydrophobic amino acids to hydrophilic amino acids. In some cases, the at least one mutation comprises changing at least ten hydrophobic amino acids to hydrophilic amino acids. In some cases, the hydrophobic amino acids can be at least one of AVILMFYW (Ala, Val, Ile, Leu, Met, Phe, Tyr, or Trp), and the hydrophilic amino acid is at least one of RHKDESTNQ (Arg, His, Lys, Asp, Glu, Ser, Thr, Asn, or Gln).

In some cases, the at least one mutation comprises changing at least one non-charged amino acid to a negatively charged amino acid. In some cases, the at least one mutation comprises changing at least two non-charged amino acids to negatively charged amino acids. In some cases, the at least one mutation comprises changing at least three non-charged amino acids to negatively charged amino acids. In some cases, the at least one mutation comprises changing at least four non-charged amino acids to negatively charged amino acids. In some cases, the noncharged amino acid is at least one of STNQCGPAVILMFYW (Ser, Thr, Asn, Gin, CYS, Gly, Pro, Ala, Val, Ile, Leu, Met, Phe, Tyr, or Trp), and the negatively charged amino acid is D or E (Asp or Glu).

In some cases, the at least one mutation comprises changing at least one positively charged amino acid to a negatively charged amino acid, wherein the positively charged amino acid is R or K, and the negatively charged amino acid is D or E. In some cases, the at least one mutation increases negative charge of caseinomacropeptide.

In some aspects, the current disclosure also provides a casein micelle, comprising a mutant κ-casein disclosed herein and at least one of αS1-casein, αS2-casein, or ß-casein. In some cases, the mutant κ-casein confers mammalian glycan-related protein functionality comprising at least one of increased solubility of the casein micelle in a liquid, increased stability of the casein micelle in a liquid, improved casein micelle formation, improved milk production, or improved curd formation in a cheese making process. In some instances, the mammalian glycan-related protein functionality comprises an improved dairy characteristic. In some cases, the increased solubility or stability is evidenced by less than 10% change in the liquid's optical density for at least a week. In some cases, the mammalian glycan-related protein functionality comprises an improved dairy characteristic.

In some aspects, the current disclosure provides a vector comprising a nucleic acid construct comprising a polynucleotide sequence encoding the mutant casein protein disclosed herein. In some cases, the vector is a recombinant retroviral vector. In some cases, the vector is a T-DNA vector. The use of plant transformation vectors comprising two separate T-DNA molecules, one T-DNA containing the gene or genes of interest (i.e., one or more insect inhibitory genes of interest) and another T-DNA containing a selectable and/or scoreable marker gene are also contemplated. In these two T-DNA vectors, the plant expression cassette or cassettes comprising the gene or genes of interest are contained within one set of T-DNA border sequences and the plant expression cassette or cassettes comprising the selectable and/or scoreable marker genes are contained within another set of T-DNA border sequences. In preferred embodiments, the T-DNA border sequences flanking the plant expression cassettes comprise both a left and a right T-DNA border sequence that are operably oriented to provide for transfer and integration of the plant expression cassettes into the plant genome. When used with a suitable Agrobacterium host in Agrobacterium-mediated plant transformation, the two T-DNA vector provides for integration of one T-DNA molecule containing the gene or genes of interest at one chromosomal location and integration of the other T-DNA containing the selectable and/or scoreable marker into another chromosomal location. Transgenic plants containing both the gene(s) of interest and the selectable and/or scoreable marker genes are first obtained by selection and/or scoring for the marker gene(s) and screened for expression of the genes of interest. Distinct lines of transgenic plants containing both the marker gene(s) and gene(s) of interest are subsequently outcrossed to obtain a population of progeny transgenic plants segregating for both the marker gene(s) and gene(s) of interest. Progeny plants containing only the gene(s) of interest can be identified by any combination of DNA RNA or protein analysis techniques. Methods for using two T-DNA vectors have been described in U.S. Pat. Nos. 6,265,638, 5,731,179, U.S. Patent Application Publication No. 2003110532A1, U.S. Patent Application Publication No. 20050183170A1, U.S. Patent No. U.S. Pat. No. 8,609,936B2, and U.S. Patent No. U.S. Pat. No. 7,884,262B2, all of which are incorporated herein by reference.

In some aspects, the current disclosure provides a food composition comprising the mutant k-casein disclosed herein. In some cases, the food composition further comprises at least one of αS1-casein, αS2-casein, or ß-casein. In some aspects, the current disclosure provides a method of making a food product, comprising expressing the mutant κ-casein disclosed herein in a plant; isolating the mutant κ-casein from the plant; and mixing the κ-casein with at least one αS1-casein, αS2-casein, or ß-casein in a solution. In some aspects, the current disclosure provides a method of making a genetically modified microorganism, wherein the microorganism is at least one of yeast, fungi, or bacteria. In some cases, the food composition comprises at least one of milk, cheese, ice cream, yogurt, cream, butter, protein powder, protein bar, and baby formula.

In some aspects, the current disclosure provides a modified casein protein comprising a modification near the C-terminus of the casein protein to increase hydrophilicity of the C-terminus region. In some instances, the modification confers mammalian glycan-related protein functionality. In some cases, the mammalian glycan-related protein functionality comprises increased solubility of the mutant casein protein in a liquid. In some cases, the mammalian glycan-related protein functionality comprises increased stability of the mutant casein protein in a liquid. In some cases, the mammalian glycan-related protein functionality comprises enhanced casein micelle formation. In some cases, the mammalian glycan-related protein functionality comprises improved milk production. In some cases, the mammalian glycan-related protein functionality comprises improved curd formation in a cheese making process. In some instances, the mammalian glycan-related protein functionality comprises an improved dairy characteristic.

In some instances, the modification comprises adding a peptide sequence to the C-terminus of the casein protein. In some cases, the added peptide sequence has at least two hydrophilic amino acids, at least three hydrophilic amino acids, at least four hydrophilic amino acids, at least five hydrophilic amino acids, at least six hydrophilic amino acids, at least seven hydrophilic amino acids, at least eight hydrophilic amino acids, at least nine hydrophilic amino acids, at least ten hydrophilic amino acids, at least 11 hydrophilic amino acids, at least 12 hydrophilic amino acids, at least 13 hydrophilic amino acids, at least 14 hydrophilic amino acids, at least 15 hydrophilic amino acids, at least 16 hydrophilic amino acids, at least 17 hydrophilic amino acids, at least 18 hydrophilic amino acids, at least 19 hydrophilic amino acids, or at least 20 hydrophilic amino acids. In some instances, the hydrophilic amino acid is at least one of R, H, K DE, S, T, N or Q. In some instances, the added peptide sequence is SDYKDDDDKHHHHHHHDE (SEQ ID No. 9). In some instances, the added peptide sequence is at least 80% identical to SEQ ID No. 9. In some instances, the added peptide sequence is at least 85% identical to SEQ ID No. 9. In some instances, the added peptide sequence is at least 90% identical to SEQ ID No. 9. In some instances, the added peptide sequence is at least 95% identical to SEQ ID No. 9. In some instances, the added peptide sequence is at least 99% identical to SEQ ID No. 9.

In some instances, the modification comprises mutating at least one hydrophobic amino acid to a hydrophilic amino acid near the C-terminus of the casein protein. In some instances, the modification comprises mutating at least two hydrophobic amino acids to hydrophilic amino acids near the C-terminus of the casein protein. In some instances, the modification comprises mutating at least three hydrophobic amino acids to hydrophilic amino acids near the C-terminus of the casein protein. In some instances, the modification comprises mutating at least four hydrophobic amino acids to hydrophilic amino acids near the C-terminus of the casein protein. In some instances, the modification comprises mutating at least five hydrophobic amino acids to hydrophilic amino acids near the C-terminus of the casein protein. In some instances, the modification comprises mutating at least six hydrophobic amino acids to hydrophilic amino acids near the C-terminus of the casein protein. In some instances, the modification comprises mutating at least eight hydrophobic amino acids to hydrophilic amino acids near the C-terminus of the casein protein. In some instances, the modification comprises mutating at least nine hydrophobic amino acids to hydrophilic amino acids near the C-terminus of the casein protein. In some instances, the modification comprises mutating at least ten hydrophobic amino acids to hydrophilic amino acids near the C-terminus of the casein protein. In some instances, the modification comprises mutating at least 11 hydrophobic amino acids to hydrophilic amino acids near the C-terminus of the casein protein. In some instances, the modification comprises mutating at least 12 hydrophobic amino acids to hydrophilic amino acids near the C-terminus of the casein protein. In some instances, the modification comprises mutating at least 13 hydrophobic amino acids to hydrophilic amino acids near the C-terminus of the casein protein. In some instances, the modification comprises mutating at least 14 hydrophobic amino acids to hydrophilic amino acids near the C-terminus of the casein protein. In some instances, the modification comprises mutating at least 15 hydrophobic amino acids to hydrophilic amino acids near the C-terminus of the casein protein. In some instances, the modification comprises mutating at least 16 hydrophobic amino acids to hydrophilic amino acids near the C-terminus of the casein protein. In some instances, the modification comprises mutating at least 17 hydrophobic amino acids to hydrophilic amino acids near the C-terminus of the casein protein. In some instances, the modification comprises mutating at least 18 hydrophobic amino acids to hydrophilic amino acids near the C-terminus of the casein protein. In some instances, the modification comprises mutating at least 19 hydrophobic amino acids to hydrophilic amino acids near the C-terminus of the casein protein. In some instances, the modification comprises mutating at least 20 hydrophobic amino acids to hydrophilic amino acids near the C-terminus of the casein protein. In some instances, the hydrophobic amino acid is at least one of A, V, I, L, M, F, Y, or W, and the hydrophilic amino acid is at least one of R, H, K, D, E, S, T, N, or Q.

In some cases, the modification comprises removing at least one hydrophobic amino acid near the C-terminus of the casein protein. In some cases, the modification comprises removing at least two hydrophobic amino acids near the C-terminus of the casein protein. In some cases, the modification comprises removing at least three hydrophobic amino acids near the C-terminus of the casein protein. In some cases, the modification comprises removing at least four hydrophobic amino acids near the C-terminus of the casein protein. In some cases, the modification comprises removing at least five hydrophobic amino acids near the C-terminus of the casein protein. In some cases, the modification comprises removing at least six hydrophobic amino acids near the C-terminus of the casein protein. In some cases, the modification comprises removing at least eight hydrophobic amino acids near the C-terminus of the casein protein. In some cases, the modification comprises removing at least nine hydrophobic amino acids near the C-terminus of the casein protein. In some cases, the modification comprises removing at least ten hydrophobic amino acids near the C-terminus of the casein protein. In some cases, the modification comprises removing at least 11 hydrophobic amino acids near the C-terminus of the casein protein. In some cases, the modification comprises removing at least 12 hydrophobic amino acids near the C-terminus of the casein protein. In some cases, the modification comprises removing at least 13 hydrophobic amino acids near the C-terminus of the casein protein. In some cases, the modification comprises removing at least 14 hydrophobic amino acids near the C-terminus of the casein protein. In some cases, the modification comprises removing at least 15 hydrophobic amino acids near the C-terminus of the casein protein. In some cases, the modification comprises removing at least 16 hydrophobic amino acids near the C-terminus of the casein protein. In some cases, the modification comprises removing at least 17 hydrophobic amino acids near the C-terminus of the casein protein. In some cases, the modification comprises removing at least 18 hydrophobic amino acids near the C-terminus of the casein protein. In some cases, the modification comprises removing at least 19 hydrophobic amino acids near the C-terminus of the casein protein. In some cases, the modification comprises removing at least 20 hydrophobic amino acids near the C-terminus of the casein protein. In some cases, the hydrophobic amino acid removed is at least one of A, V, I, L, M, F, Y, or W.

In some instances, the modified casein protein is a kappa casein. In some cases, the modified casein protein is a bovine casein. In some cases, the modified casein protein is a ruminant casein. In some cases, the modified casein protein is a human casein. In some cases, the modified casein protein is non-glycosylated, under-glycosylated, or differentially glycosylated.

In some aspects, the current disclosure provides a casein micelle, comprising any one of the modified caseins disclosed herein. In some instances, the casein micelle comprises a modified casein which is a κ-casein, and further comprises at least one of αS1-casein, αS2-casein, or ß-casein. In some instances, the modified κ-casein enhances the functionality of the casein micelle. In some instances, the enhanced functionality comprises increased stability or solubility of the casein micelle, or improved curd formation in a cheese making process using the casein micelle.

In some aspects, the current disclosure provides a food composition comprising any one of the modified caseins disclosed herein. In some aspects, the current disclosure provides a food composition comprising any one of the casein micelles disclosed herein. In some instances, the food composition comprises at least one of milk, cheese, ice cream, yogurt, cream, butter, protein powder, protein bar, and baby formula.

In some aspects, the current disclosure provides a method of making a casein micelle in vitro, comprising expressing a modified casein protein disclosed herein, wherein the modified casein protein is κ-casein; isolating the modified κ-casein from the plant; and mixing the modified κ-casein with at least one of αS1-casein, αS2-casein, or ß-casein in a solution, thereby forming the casein micelle.

In some aspects, the current disclosure provides method of making a casein micelle in vitro, comprising expressing a modified casein protein disclosed herein in a microorganism; wherein the modified casein protein is κ-casein; isolating the modified κ-casein from the microorganism; and mixing the modified κ-casein with at least one of αS1-casein, αS2-casein, or ß-casein in a solution, thereby forming the casein micelle.

In some aspects, the current disclosure provides a method of making a casein micelle in vivo, comprising co-expressing in a plant or microorganism, 1) a modified casein protein disclosed herein, wherein the modified casein protein is κ-casein, and 2) at least one of αS1-casein, αS2-casein, or ß-casein, wherein the modified κ-casein and at least one of αS1-casein, αS2-casein, or ß-casein form the casein micelle in vivo. In some cases, the plant is soybean. In some cases, the microorganism is a fugus (e.g., yeast) or bacteria.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1 shows a non-limiting example of a vector for expressing proteins in a plant providing mammalian glycan-related protein functionality.

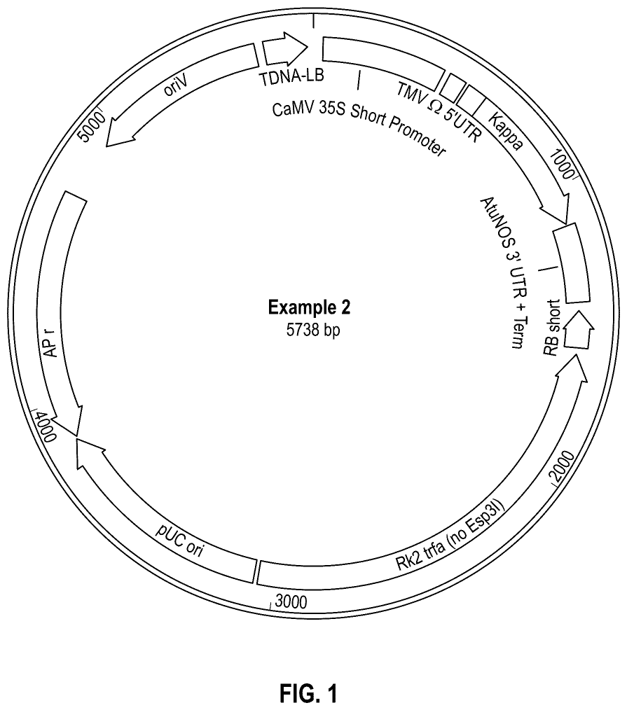

FIG. 2 shows a non-limiting example of a vector for expressing proteins in a plant providing mammalian glycan-related protein functionality, where kappa casein has three amino acids mutated: I to N, I to Q, and I to K.

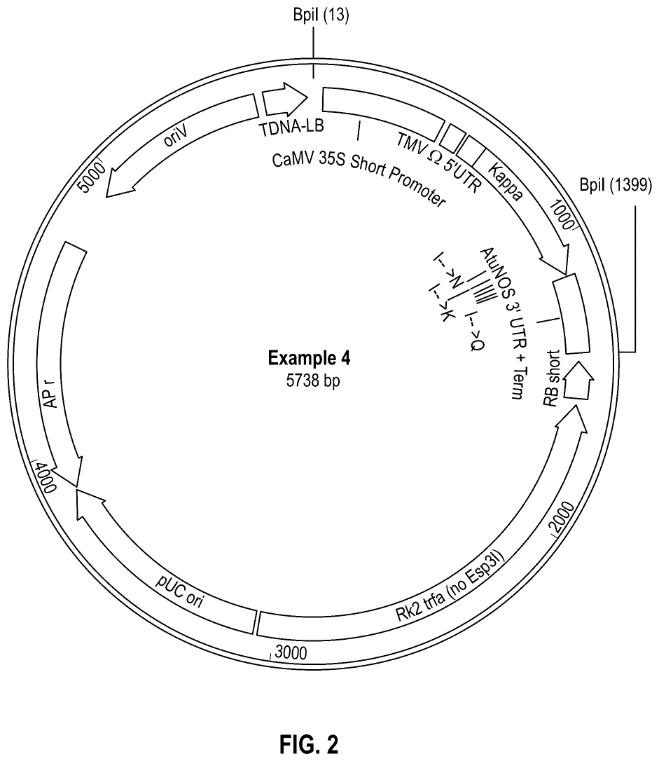

FIG. 3 shows a non-limiting example of a vector for expressing proteins in a plant providing mammalian glycan-related protein functionality, where kappa casein has two amino acids mutated: A to R and V to Q.

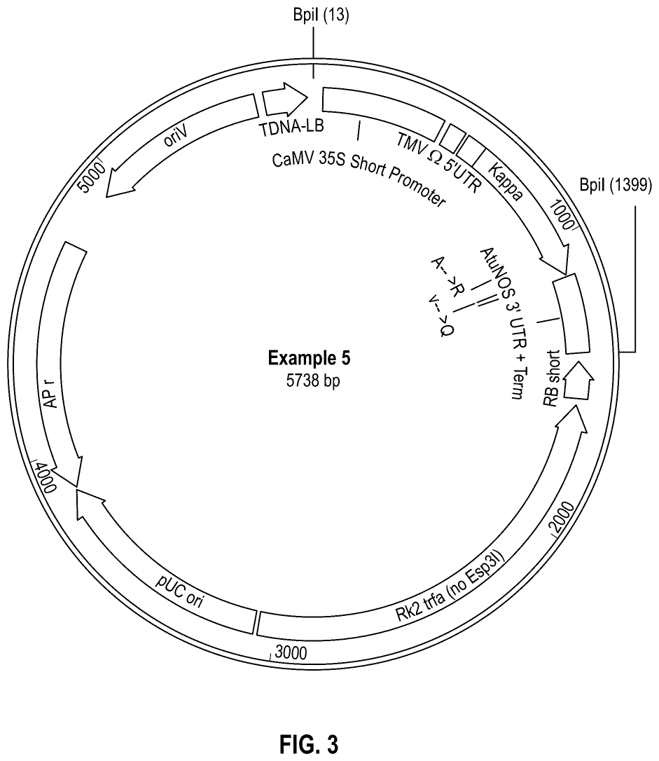

FIG. 4 shows a non-limiting example of a vector for expressing proteins in a plant providing mammalian glycan-related protein functionality, where kappa casein has three amino acids mutated: I to D, V to R, and I to K.

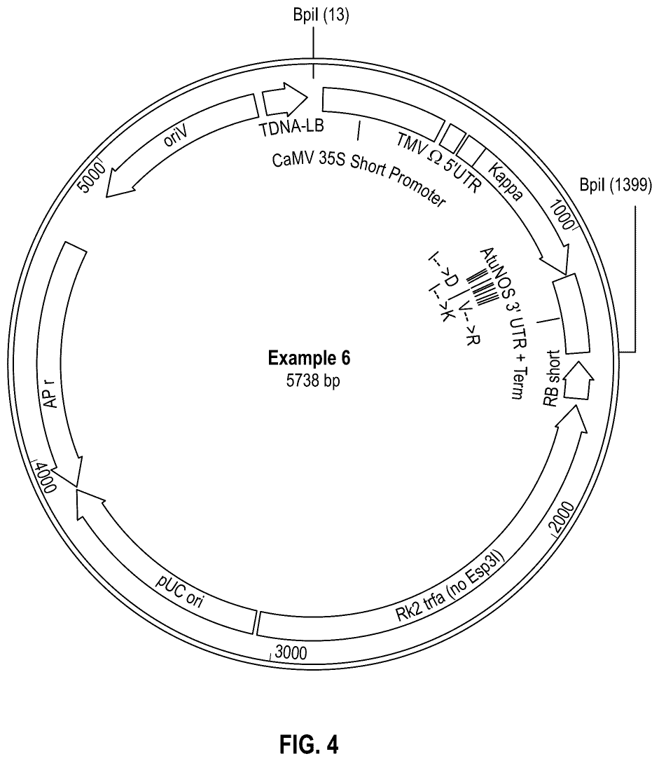

FIG. 5 shows a non-limiting example of a vector for expressing proteins in a plant providing mammalian glycan-related protein functionality, where kappa casein has two amino acids mutated: Q to E and S to Q.

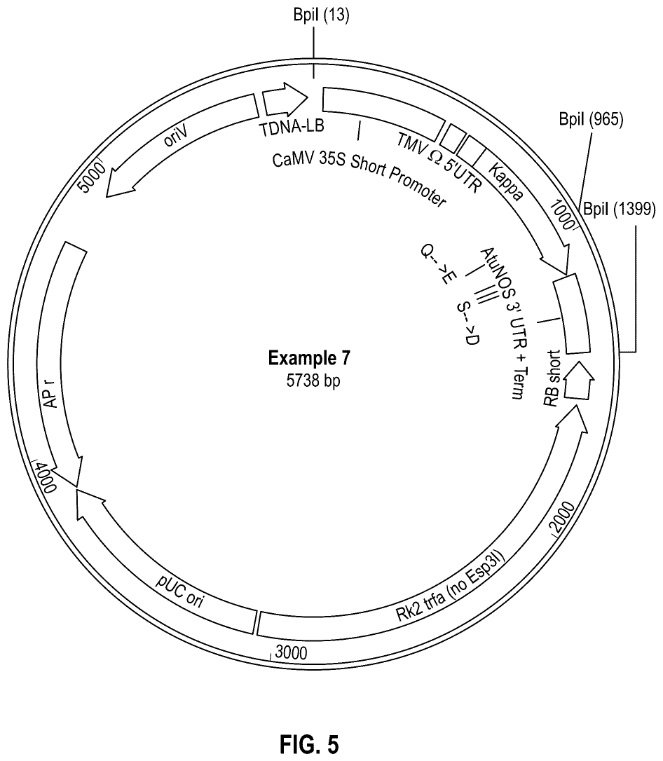

FIG. 6 shows a non-limiting example of a vector for expressing proteins in a plant providing mammalian glycan-related protein functionality, where kappa casein has one amino acid mutated: K to D.

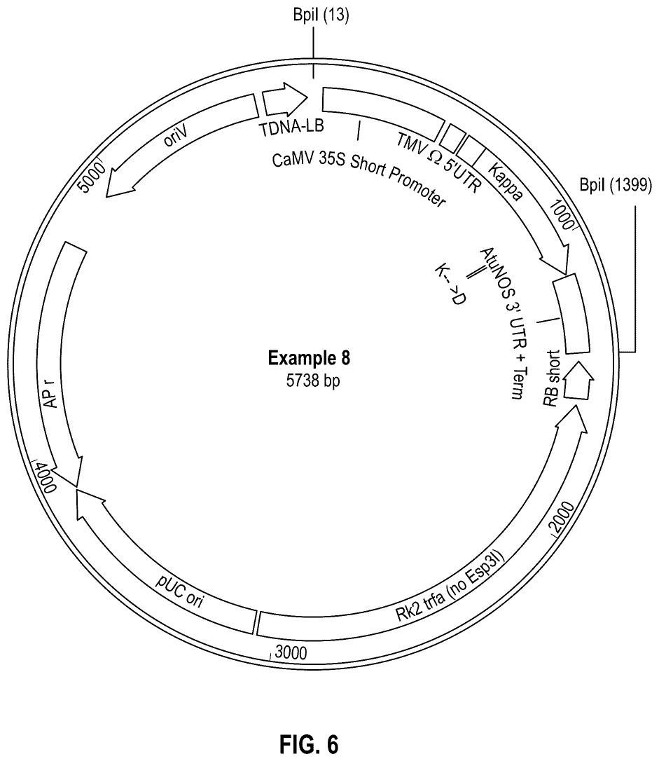

FIG. 7 shows a non-limiting example of a vector for expressing proteins in a plant providing mammalian glycan-related protein functionality, where kappa casein has three amino acids mutated: Q to E, T to E, and S to D.

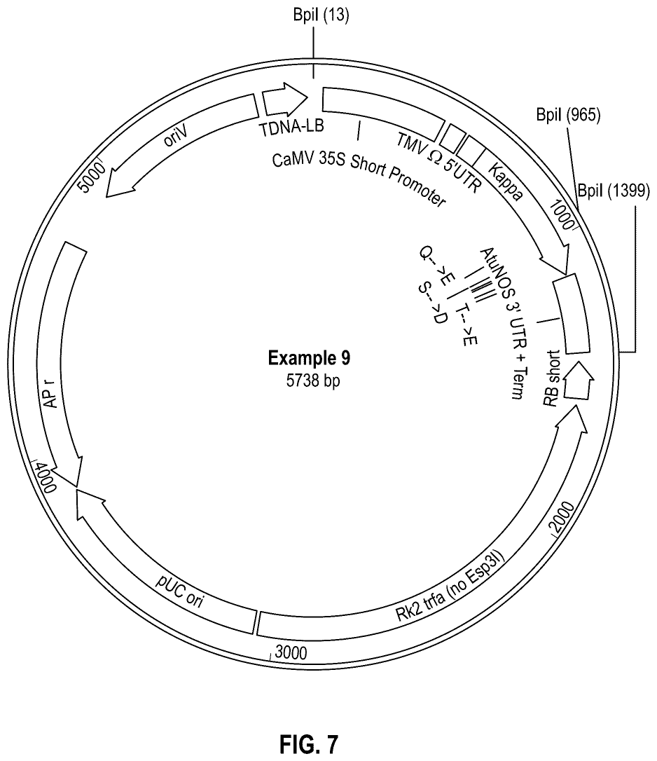

FIG. 8 shows cheese formation in Example 4. FIG. 8A shows precipitate formed around the edge of the beaker, indicating successful formation of a cheese matrix. FIG. 8B, FIG. 8C and FIG. 8D show cheese collected exhibiting typical cheese color and texture. FIG. 8E shows milk made using the methods described in Example 4.

FIG. 9A shows casein proteins expressed in a plant using methods described in Example 4. FIG. 9B is an enlarged view of a portion of FIG. 9A.

FIG. 10 shows casein micelles formed using methods described in Example 4.

FIG. 11 shows the structure of the vector used in Example 4.

### DETAILED DESCRIPTION

The following embodiments are described in sufficient detail to enable those skilled in the art to make and use the invention. It is to be understood that other embodiments would be evident based on the present disclosure, and that system, process, or mechanical changes can be made without departing from the scope of an embodiment of the present disclosure.

In the following description, numerous specific details are given to provide a thorough understanding of the invention. However, it will be apparent that the invention can be practiced without these specific details. In order to avoid obscuring an embodiment of the present disclosure, some well-known techniques, system configurations, and process steps are not disclosed in detail. Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

### Definitions

These and other valuable aspects of the embodiments of the present disclosure consequently further the state of the technology to at least the next level. While the disclosure has been described in conjunction with a specific best mode, it is to be understood that many alternatives, modifications, and variations will be apparent to those skilled in the art in light of the descriptions herein. Accordingly, it is intended to embrace all such alternatives, modifications, and variations that fall within the scope of the included claims. All matters set forth herein or shown in the accompanying drawings are to be interpreted in an illustrative and non-limiting sense.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.’

Use of absolute or sequential terms, for example, “will,” ‘will not,” “shall,” “shall not,’ “xmust,” “must not,” “first,′ initially,” “next,” subsequently,” “before,’ after, “lastly,” and “finally,” are not meant to limit scope of the present embodiments disclosed herein but as exemplary.

As used herein, “or” may refer to “and”, “or,” or “and/or” and may be used both exclusively and inclusively. For example, the term “A or B” may refer to “A or B”, “A but not B” “B but not A”, and “A and B” In some cases, context may dictate a particular meaning.

Any systems, methods, software, and platforms described herein are modular and not limited to sequential steps. Accordingly, terms such as “first” and “second” do not necessarily imply priority, order of importance, or order of acts.

As used herein, the term “about” or the symbol “Q” when referring to a number or a numerical range means that the number or numerical range referred to is an approximation within experimental variability (or within statistical experimental error), and the number or numerical range may vary from, for example, from 1% to 10% of the stated number or numerical range. Unless otherwise indicated by context, the term “about” refers to +10% of a stated number or value.

As used herein, the term “approximately” means within an acceptable error range for the particular value as determined by one of ordinary skill in the art, which will depend in part on how the value is measured or determined, e.g., the limitations of the measurement system. For example, “approximately” can mean within 1 or more than 1 standard deviation, per the practice in the given value. Where particular values are described in the application and claims, unless otherwise stated the term “approximately” should be assumed to mean an acceptable error range for the particular value.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and the like. All language such as «up to,” “at least,” “greater than,” “less than,” and the like include the number recited and refer to ranges which can be subsequently broken down into sub-ranges as discussed above. Finally, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Whenever the term “at least,” “greater than,” “greater than or equal to”, or a similar phrase precedes the first numerical value in a series of two or more numerical values, the term “at least,” “greater than,” “greater than or equal to” or similar phrase applies to each of the numerical values in that series of numerical values. For example, “at least 1, 2, or 3” is equivalent to “at least 1, at least 2, and/or at least 3.”

Whenever the term “no more than,” “less than,” “less than or equal to,” “no greater than, “at most” or a similar phrase, precedes the first numerical value in a series of two or more numerical values, the term “no more than,” “less than,” “less than or equal to,” “no greater than,”, “at most,” or similar phrase applies to each of the numerical values in that series of numerical values. For example, “less than 3, 2, or 1 is equivalent to “less than 3, less than 2, and/or less than 1.”

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality” and “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “consisting essentially of’ refers to the inclusion of recited elements and other elements that do not materially affect the basic and novel characteristics of a claimed combination.

As used herein, a “vector” is a plasmid comprising operably linked polynucleotide sequences that facilitate expression of a coding sequence in a particular host organism (e.g., a bacterial expression vector or a plant expression vector). Polynucleotide sequences that facilitate expression in prokaryotes can include, e.g., a promoter, an enhancer, an operator, and a ribosome binding site, often along with other sequences. Eukaryotic cells can use promoters, enhancers, termination and polyadenylation signals and other sequences that are generally different from those used by prokaryotes.

As used herein, the term “plant” includes whole plant, plant organ, plant tissues, and plant cell and progeny of same, but is not limited to angiosperms and gymnosperms such as Arabidopsis, potato, tomato, tobacco, alfalfa, lemice, carrot, strawberry, sugarbeet, cassava, sweet potato, soybean, lima bean, pea, chick pea, maize (com), turf grass, wheat, rice, barley, sorghum, oat, oak, eucalyptus, walnut, palm and duckweed a well as fern and moss. Thus, a plant may be a monocot, a dicot, a vascular plant reproduced from spores such as fern or a nonvascular plant such as moss, liverwort, hornwort and algae. The term “plant,” as used herein, also encompasses plant cells, seeds, plant progeny, propagule whether generated sexually or asexually, and descendants of any of these, such as cuttings or seed. Plant cells include suspension cultures, callus, embryos, meristematic regions, callus tissue, leaves, roots, shoots, gametophytes, sporophytes, pollen, seeds and microspores. Plants may be at various stages of maturity and may be grown in liquid or solid culture, or in soil or suitable media in pots, greenhouses or fields.

As used herein, the term “dicot” refers to a flowering plant whose embryos have two seed leaves or cotyledons. Examples of dicots include Arabidopsis, tobacco, tomato, potato, sweet potato, cassava, alfalfa, lima bean, pea, chick pea, soybean, carrot, strawberry, lettuce, oak, maple, walnut, rose, mint, squash, daisy, quinoa, buckwheat, mung bean, cow pea, lentil, lupin, peanut, fava bean, French beans, mustard, or cactus.

As used herein, the term “monocot” refers to a flowering plant whose embryos have one cotyledon or seed leaf. Examples of monocots include turf grass, maize (corn), rice, oat, wheat, barley, sorghum, orchid, iris, lily, onion, palm, and duckweed.

As used herein, the term “transgenic plant” means a plant that has been transformed with one or more exogenous nucleic acids. “Transformation” refers to a process by which a nucleic acid is stably integrated into the genome of a plant cell. “Stably transformed” refers to the permanent, or non-transient, retention, expression, or a combination thereof of a polynucleotide in and by a cell genome. A stably integrated polynucleotide is one that is a fixture within a transformed cell genome and can be replicated and propagated through successive progeny of the cell or resultant transformed plant. Transformation can occur under natural or artificial conditions using various methods. Transformation can rely on any method for the insertion of nucleic acid sequences into a prokaryotic or eukaryotic host cell, including Agrobacterium-mediated transformation as illustrated in U.S. Pat. Nos. 5,159,135; 5,824,877; 5,591,616; 6,384,301, and all of which are incorporated herein by reference in its entirety. Methods for plant transformation also include microprojectile bombardment as illustrated in U.S. Pat. Nos. 5,015,580; 5,550,318; 5,538,880; 6,153,812; 6,160,208; 6,288,312 and 6,399,861, all of which are incorporated herein by reference in its entirety. Recipient cells for the plant transformation include meristem cells, callus, immature embryos, hypocotyls explants, cotyledon explants, leaf explants, and gametic cells such as microspores, pollen, sperm and egg cells, and any cell from which a fertile plant can be regenerated, as described in U.S. Pat. Nos. 6,194,636; 6,232,526; 6,541,682; and 6,603,061 and U.S. Patent Application publication US 2004/0216189 A1, all of which are incorporated herein by reference in its entirety.

As used herein, the term “stably expressed” refers to expression and accumulation of a protein in a plant cell over time. As an example, a recombinant protein may accumulate because it is not degraded by endogenous plant proteases. As a further example, a recombinant protein is considered to be stably expressed in a plant if it is present in the plant in an amount of 1% or higher per total protein weight of soluble protein extractable from the plant.

As used herein, the term “recombinant” refers to nucleic acids or proteins formed by laboratory methods of genetic recombination (e.g., molecular cloning) to bring together genetic material from multiple sources, creating sequences that would otherwise not be found in the genome. Recombinant proteins may be expressed in vivo in various types of host cells, including plant cells, bacterial cells, fungal cells, avian cells, and mammalian cells. Recombinant proteins may also be generated in vitro. As used herein, the term “tagged protein” refers to a recombinant protein that includes additional peptides that are not part of the native protein and that remain after post-translational processing.

As used herein, the term “casein micelles” means micelles comprising casein proteins. Examples of casein micelles are described in U.S. patent application Ser. No. 16/741,680 (Patent No. U.S. Pat. No. 11,326,176), filed on Jan. 13, 2020, titled “Recombinant micelle and method of in vivo assembly,” and in U.S. patent application Ser. No. 17/826,021 filed on May 26, 2022, both incorporated herein by reference in its entirety. Recombinant casein micelles can be made in vivo or in vitro using the methods described therein. United States patent application U.S. Ser. No. 17/826,021 (United States Patent Application US20220290167A1), titled “Recombinant micelle and method of in vivo assembly” teaches vectors and sequences for making recombinant casein proteins and micelles, which is incorporated herein by reference in its entirety.

U.S. Pat. No. 11,457,649 describes a substitute dairy food, and U.S. patent application Ser. No. 16/862,011 (Publication No. US20210010017A1) describes food compositions comprising a milk protein, both of which are incorporated herein by reference in their entirety.

Where a definition or use of a term in an incorporated reference is inconsistent or contrary to the definition of that term provided herein, the definition of that term provided herein applies and the definition of that term in the reference does not apply.

As used herein, the term “milk” means a liquid composition that contains soluble casein micelles and where the weight of soluble casein micelles is equal to or greater than 100 of the total protein weight in the composition.

As used herein, the term “cheese curd” means a solid or semi-solid mass made by gelating, coagulating, or curdling milk.

As used herein, the term “cheese” means a food made from cheese curds.

As used herein, the term differentially-glycosylated means the absence of glycans on the protein or the glycosylation of the protein with 1 or more N or O linked glycans not commonly found on the protein in the native organism.

As used herein, the term “dairy characteristic” means a characteristic selected from one of the following characteristics of a dairy food: adhesiveness, airiness, appearance, aroma, binding, chewiness, coagulation, cohesiveness, compactness, creaminess, crispiness, crumbliness, density, elasticity, emulsification, fattiness, firmness, flavor, foaminess, graininess, greasiness, hardness, handling, juiciness, leavening, mouthcoating, mouthfeel, richness, roughness, slipperiness, smoothness, springiness, structure, taste, tenderness, texture, thickness, uniformity, and wetness. As used herein, the term “in-vitro” means outside a living organism.

As used herein, the term “fusion protein” refers to a protein comprising at least two constituent proteins that are encoded by separate genes, and that have been joined so that they are transcribed and translated as a single polypeptide.

In some cases, the mammalian glycan-related protein functionality comprises increased solubility of the mutant casein protein in a liquid. In some cases, the mammalian glycan-related protein functionality comprises increased stability of the mutant casein protein in a liquid. In some cases, the mammalian glycan-related protein functionality comprises enhanced casein micelle formation. In some cases, the mammalian glycan-related protein functionality comprises improved milk production. In some cases, the mammalian glycan-related protein functionality comprises improved curd formation in a cheese making process. In some instances, the mammalian glycan-related protein functionality comprises an improved dairy characteristic.

In some aspects, the disclosed compositions, methods and systems allow for the production of casein proteins, and in particular, functional casein proteins and casein micelles with desirable characteristics in non-animal organisms. In some cases, the non-glycosylated, partially-glycosylated, or differentially-glycosylated proteins are produced in transgenic plants. In some cases, the non-glycosylated, partially-glycosylated, or differentially-glycosylated proteins are produced in yeast, fungus, and bacteria.

In some aspects, the present disclosure relates to non-glycosylated, under-glycosylated, or differentially-glycosylated recombinant proteins and methods of improving expression, solubility, stability, and functionality of these proteins in transgenic host organisms. In some aspects, the present disclosure also relates to food compositions comprising non-glycosylated, under-glycosylated, or differentially-glycosylated recombinant proteins.

In some aspects, the current disclosure provides compositions, methods and systems for improving the functional characteristics of non-glycosylated, under-glycosylated, or differentially-glycosylated proteins in plants. In some cases, the current disclosure provides vectors for expressing proteins in a plant where hydrophobic residues are replaced with hydrophilic residues providing mammalian glycan-related protein functionality, for example, micelle formation and stability, in the absence of mammalian glycans.

In some aspects, the current disclosure provides a mutant casein protein comprising at least one mutation compared with a wild-type casein protein counterpart, wherein the at least one mutation provides mammalian glycan-related protein functionality. In some cases, the mutant casein protein is a mutated alpha-casein, beta-casein, or kappa-casein. In some cases, the at least one mutation is located on the caseinomacropeptide of the mutated κ-casein. In some cases, the at least one mutation is located on the N-terminal of the caseinomacropeptide of the mutated κ-casein. In some cases, the at least one mutation is located at C-terminal caseinomacropeptide of the mutated κ-casein.

In some cases, the at least one mutation comprises changing at least one hydrophobic amino acid to a hydrophilic amino acid. In some cases, the at least one mutation comprises changing at least two hydrophobic amino acids to hydrophilic amino acids. In some cases, the at least one mutation comprises changing at least three hydrophobic amino acids to hydrophilic amino acids. In some cases, the at least one mutation comprises changing at least six hydrophobic amino acids to hydrophilic amino acids. In some cases, the at least one mutation comprises changing at least ten hydrophobic amino acids to hydrophilic amino acids. In some cases, the hydrophobic amino acids can be at least one of AVILMFYW (Ala, Val, Ile, Leu, Met, Phe, Tyr, or Trp), and the hydrophilic amino acid is at least one of RHKDESTNQ (Arg, His, Lys, Asp, Glu, Ser, Thr, Asn, or Gln).

In some cases, the at least one mutation comprises changing at least one non-charged amino acid to a negatively charged amino acid. In some cases, the at least one mutation comprises changing at least two non-charged amino acids to negatively charged amino acids. In some cases, the at least one mutation comprises changing at least three non-charged amino acids to negatively charged amino acids. In some cases, the at least one mutation comprises changing at least four non-charged amino acids to negatively charged amino acids. In some cases, the noncharged amino acid is at least one of STNQCGPAVILMFYW (Ser, Thr, Asn, Gln, CYS, Gly, Pro, Ala, Val, Ile, Leu, Met, Phe, Tyr, or Trp), and the negatively charged amino acid is D or E (Asp or Glu).

In some cases, the at least one mutation comprises changing at least one positively charged amino acid to a negatively charged amino acid, wherein the positively charged amino acid is R or K, and the negatively charged amino acid is D or E. In some cases, the at least one mutation increases negative charge of caseinomacropeptide.

In some aspects, the current disclosure also provides a casein micelle, comprising a mutant κ-casein disclosed herein and at least one of αS1-casein, αS2-casein, or ß-casein. In some cases, the mutant κ-casein confers mammalian glycan-related protein functionality comprising at least one of increased solubility of the casein micelle in a liquid, increased stability of the casein micelle in a liquid, improved casein micelle formation, improved milk production, or improved curd formation in a cheese making process. In some cases, the increased solubility or stability is evidenced by less than 10% change in the liquid's optical density for at least a week.

In some aspects, the current disclosure provides vector comprising a nucleic acid construct comprising a polynucleotide sequence encoding the mutant casein protein disclosed herein. In some cases, the vector is a recombinant retroviral vector. In some cases, the vector is a TDNA vector.

In some aspects, the current disclosure provides a food composition comprising the mutant κ-casein disclosed herein. In some cases, the food composition further comprises at least one of αS1-casein, αS2-casein, or ß-casein. In some aspects, the current disclosure provides a method of making a food product, comprising expressing the mutant κ-casein disclosed herein in a plant; isolating the mutant κ-casein from the plant; and mixing the κ-casein with at least one of αS1-casein, αS2-casein, or ß-casein in a solution. In some aspects, the current disclosure provides a method of making a genetically modified microorganism, wherein the microorganism is at least one of yeast, fungi, or bacteria. In some cases, the food composition comprises at least one of milk, cheese, ice cream, yogurt, cream, butter, protein powder, protein bar, and baby formula.

In some aspects, the current disclosure also provides food compositions comprising the composition disclosed herein or made using the methods disclosed herein. Contemplated food compositions include dairy products or products that resembles a dairy product (i.e., dairy product substitutes). The term “dairy product” as used herein refers to milk (e.g., whole milk (at least 3.25% milk fat), partly skimmed milk (from 1% to 2% milk fat), skim milk (less than 0.2% milk fat), cooking milk, condensed milk, flavored milk, goat milk, sheep milk, dried milk, evaporated milk, milk foam), and products derived from milk, including but not limited to yogurt (e.g., whole milk yogurt (at least 6 grams of fat per 170 g), low-fat yogurt (between 2 and 5 grams of fat per 170 g), nonfat yogurt (0.5 grams or less of fat per 170 g), greek yogurt (strained yogurt with whey removed), whipped yogurt, goat milk yogurt, Labneh (labne), sheep milk yogurt, yogurt drinks (e.g., whole milk Kefir, low-fat milk Kefir), Lassi), cheese (e.g., whey cheese such as ricotta; pasta filata cheese such as mozzarella; semi-soft cheese such as Havarti and Muenster; medium-hard cheese such as Swiss and Jarlsberg; hard cheese such as Cheddar and Parmesan; washed curd cheese such as Colby and Monterey Jack; soft ripened cheese such as Brie and Camembert; fresh cheese such as cottage cheese, feta cheese, cream cheese, and curd; processed cheese; processed cheese food; processed cheese product; processed cheese spread; enzyme-modulated cheese; cold-pack cheese), dairy-based sauces (e.g., fresh, frozen, refrigerated, or shelf stable), dairy spreads (e.g., low-fat spread, low-fat butter), cream (e.g., dry cream, heavy cream, light cream, whipping cream, half-and-half, coffee whitener, coffee creamer, sour cream, creme fraiche), frozen confections (e.g., ice cream, smoothie, milk shake, frozen yogurt, sundae, gelato, custard), dairy desserts (e.g., fresh, refrigerated, or frozen), butter (e.g., whipped butter, cultured butter), dairy powders (e.g., whole milk powder, skim milk powder, fat-filled milk powder (i.e., milk powder comprising plant fat in place of all or some animal fat), infant formula, milk protein concentrate (i.e., protein content of at least 80% by weight), milk protein isolate (i.e., protein content of at least 90% by weight), whey protein concentrate, whey protein isolate, demineralized whey protein concentrate, demineralized whey protein concentrate, .beta.-lactoglobulin concentrate, .beta.-lactoglobulin isolate, .alpha.lactalbumin concentrate, .alpha.-lactalbumin isolate, glycomacropeptide concentrate, glycomacropeptide isolate, casein concentrate, casein isolate, nutritional supplements, texturizing blends, flavoring blends, coloring blends), ready-to-drink or ready-to-mix products (e.g., fresh, refrigerated, or shelf stable dairy protein beverages, weight loss beverages, nutritional beverages, sports recovery beverages, and energy drinks), puddings, gels, chewables, crisps, and bars. The term “food product substitute” (e.g., “dairy product substitute”) as used herein refers to a food product that resembles a conventional food product (e.g., can be used in place of the conventional food product). Such resemblance can be due to any physical, chemical, or functional attribute. In some embodiments, the resemblance of the food product provided herein to a conventional food product is due to a physical attribute. Non-limiting examples of physical attributes include color, shape, mechanical characteristics (e.g., hardness, G′ storage modulus value, shape retention, cohesion, texture (i.e., mechanical characteristics that are correlated with sensory perceptions (e.g., mouthfeel, fattiness, creaminess, homogenization, richness, smoothness, thickness), viscosity, and crystallinity. In some embodiments, the resemblance of the food product provided herein and a conventional food product is due to a chemical/biological attribute. Non-limiting examples of chemical attributes include nutrient content (e.g., type and/or amount of amino acids (e.g., PDCAAS score), type and/or amount of lipids, type and/or amount of carbohydrates, type and/or amount of minerals, type and/or amount of vitamins), pH, digestibility, shelf-life, hunger and/or satiety regulation, taste, and aroma. In some embodiments, the resemblance of the food product provided herein to a conventional food product is due to a functional attribute. Nonlimiting examples of functional attributes include gelling/agglutination behavior (e.g., gelling capacity (i.e., time required to form a gel (i.e., a protein network with spaces filled with solvent linked by hydrogen bonds to the protein molecules) of maximal strength in response to a physical and/or chemical condition (e.g., agitation, temperature, pH, ionic strength, protein concentration, sugar concentration, ionic strength), agglutination capacity (i.e., capacity to form a precipitate (i.e., a tight protein network based on strong interactions between protein molecules and exclusion of solvent) in response to a physical and/or chemical condition), gel strength (i.e., strength of gel formed, measured in force/unit area (e.g., pascal (Pa)), water holding capacity upon gelling, syneresis upon gelling (i.e., water weeping over time), foaming behavior (e.g., foaming capacity (i.e., amount of air held in response to a physical and/or chemical condition), foam stability (i.e., half-life of foam formed in response to a physical and/or chemical condition), foam seep), thickening capacity, use versatility (i.e., ability to use the food product in a variety of manners and/or to derive a diversity of other compositions from the food product; e.g., ability to produce food products that resemble milk derivative products such as yoghurt, cheese, cream, and butter), and ability to form protein dimers.

### Example 1. Assay for Probing the Functionality of Kappa Casein

1. Individual caseins are mixed together in a particular ratio (kappa casein: beta casein: alpha casein=6:16:27).
2. Slowly add CaCl2 and Na3PO4 in small aliquots over time while stirring to initiate micelle formation.
3. White colloid forms.
4. Measure OD600 of the resulting white colloid.
5. Incubate the sample in 4° C. for 1 week.
6. Measure OD600 again to assess the stability of the micelles.

Higher OD readings correspond to more casein in micellar form. As the micelles precipitate out of solution the OD measurement drops. The longer the sample shows a high OD reading the more functional kappa casein is in stabilizing the micelles.

### Example 2 Casein from Soybean Embryos

This method can be used for the production of casein from soybean embryos, which has potential applications in the food industry as a source of plant-based protein.

Method for Production of Casein from Soybean Embryos:
1 Transformation: Transform soybean embryos with a plasmid containing casein genes (experimental) or a plasmid without casein genes (control).
2. Protein extraction: Extract total protein from transformed soybean embryos using Tris Extraction Buffer.
3. Purification: Purify caseins from the total protein using Nickel-NTA resin.
4. Elution: Elute casein from the resin using an imidazole-containing buffer.
5. Concentration: Concentrate the purified casein using a dialysis spin column.
6. Calcium and phosphate addition: Add CaCl2 and Na3PO4 to the concentrated casein samples.
7. Optical density measurement: Measure the optical density (OD) of the casein samples at a wavelength of 600 nm.
8. Comparison: Compare the OD values of the experimental and control samples.

Expected result: The experimental samples will have a higher OD value than the control samples due to the presence of casein micelles.

### Example 3. Cheese from Recombinant Kappa

Transformation of E. Coli: E. Coli cells were transformed with a plasmid containing a gene encoding for kappa casein, along with a selection marker, using standard methods.

Culture conditions: The transformed E. Coli cells were grown in a suitable medium at 37° C. with shaking until the culture reached an optical density of 0.6-0.8 at 600 nm.

Induction of gene expression: Expression of the kappa casein gene was induced by adding 0.5 mM isopropyl β-D-1-thiogalactopyranoside (IPTG) to the culture and incubating for a further 4 hours.

Harvesting of cells: The cells were harvested by centrifugation at 4,000g for 15 minutes.

Cell lysis: The cell pellet was resuspended in lysis buffer (50 mM Tris-HCl, 150 mM NaCl, 1 mM EDTA, 1 mM phenylmethylsulfonyl fluoride (PMSF), pH 7.5) and lysed using a French press or sonication.

Purification of kappa casein: The lysate was clarified by centrifugation at 20,000g for 30 minutes at 4° C. The resulting supernatant was loaded onto a cation exchange chromatography column equilibrated with binding buffer (20 mM sodium phosphate, 150 mM NaCl, pH 7.0). Kappa casein was eluted using a linear gradient of 0-1M NaCl in binding buffer. Fractions containing kappa casein were pooled and concentrated using an ultrafiltration device.

Verification of purity: The purity of the kappa casein was verified by SDS-PAGE and western blotting using anti-kappa casein antibodies.

Kappa casein transferred to a 15 mL tube. A 100 mM NaCl solution and a 10 mM Tris buffer at pH 7.5 were prepared and 5 mL of this solution was added to the tube with the pellets. The mixture was extensively vortexed to dissolve the pellets and the resulting solution was concentrated to a final concentration of 0.5 mg/mL using an ultrasonicator. The ultrasonication was performed for 5 minutes at 6 watts with the probe immersed deeply in the solution.

After letting the solution settle for 10 minutes, the concentration was measured and found to be 10 mg/mL. A 1 mL sample of this solution was transferred to a 5 ml beaker and 27.7 mg of Sigma alpha casein and 15.8 mg of Sigma beta casein were added to the beaker. The mixture was stirred at 37° C. until the casein was completely dissolved.

The typical 2 mL milk/cheese protocol was then performed. The pH of the solution was

5.2, and 30 μL of citric acid was added to adjust the pH. The temperature of the solution was maintained at 14° C. throughout the experiment. The typical 2 mL milk/cheese protocol:

Tripotassium Citrate is prepared by mixing 0.324g Potassium citrate tribasic and 1.5 mL water. Potassium phosphate is prepared by mixing 0.383g K2HPO4 and 11 mL water. Calcium chloride is prepared by mixing 0.470g CaCl2-2H2O and 15 mL water. Add 1 mL of casein containing water to a 5 ml beaker. Add a mini stir bar. Place the 5 mL beaker inside of a 600 ml beaker on a hot plate containing approximately 125 ml of water to submerge the 5 ml beaker about a third of the height, using a clamping system (shown at the bottom of the page). Make sure the 5 mL beaker is not touching the bottom or the sides of the 600 mL beaker. Place a thermometer in the outer water with the bulb not touching the glass but fully submerged in the water. Set the hotplate around 65° C. after all of the casein is in solution adjust accordingly to make sure the water temperature stays at 37° C. Try to get the water temperature to 37C quickly to avoid too much evaporation. Check the temperature on the thermometer periodically. Set the stirring to 1000 RPM. Add 20 uL tripotassium citrate and 70 uL potassium phosphate. Wait 4 minutes, then every 4 minutes add 12.5 uL Potassium Phosphate Solution and 25 uL Calcium Chloride Sol for a total of 12 times. Stir with the temperature at 37° C. for 1 hour then turn the heat off. Add 240 uL water and 180 uL Heavy Cream.

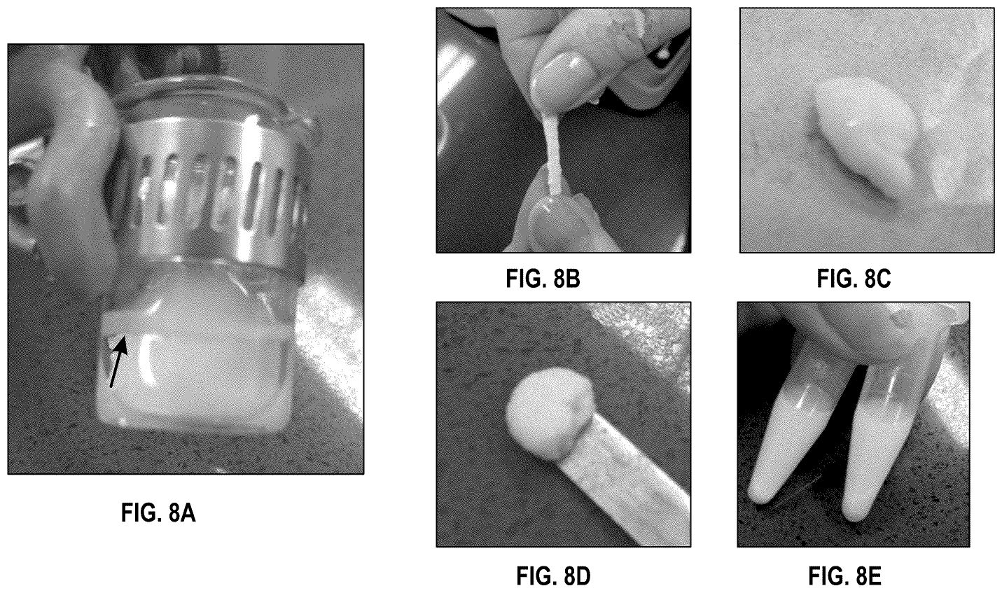

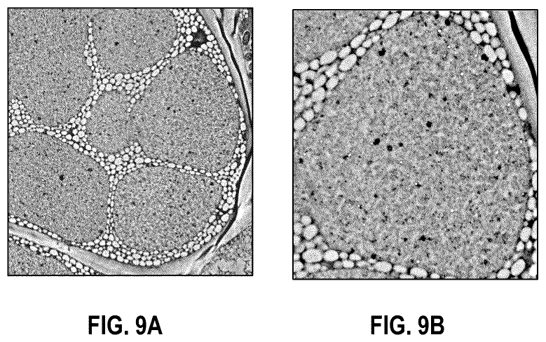

Result: precipitate was formed around the edge of the beaker, indicating successful formation of a cheese matrix (FIG. 8). The formed cheese is collected (FIG. 9), exhibiting traditional cheese color and texture.

### Example 4: Casein Micelles from Non-Glycosylated, Under-Glycosylated, or Differentially glycosylated caseins

In this experiment we demonstrate that increasing the hydrophilicity of the C-terminus region of κ-casein induces micelle formation of differentially glycosylated κ-casein.

Vectors: A multigene vector, termed pMOZ1066, which expresses the coding regions 1) bovine α-S1-casein (Uniprot accession #P02662) (SEQ ID No. 1), 2) green fluorescent protein (GFP, Uniprot accession #P42212) (SEQ ID No. 2), 3) bovine ß-casein (Uniprot accession #P02666) (SEQ ID No. 3), 4) bovine κ-casein (Uniprot accession #P02668) (SEQ ID No. 4), and 5) bovine FAM20C kinase (uniprot accession number #FIMXQ3) (SEQ ID No. 5) was assembled using the modular cloning system MoClo (Engler, Carola, Mark Youles, Ramona Gruetzner, Tim-Martin Ehnert, Stefan Werner, Jonathan D. G. Jones, Nicola J. Patron, and Sylvestre Marillonnet. “A Golden Gate Modular Cloning Toolbox for Plants.” ACS Synthetic Biology 3, no. 11 (Nov. 21, 2014): 839-43. https://doi.org/10.1021/sb4001504). All five proteins were expressed under constitutively active plant promoters, with a subset of these proteins possessing translationally-fused epitope tags and/or target-peptide sequences on their C-termini. Specifics on each gene subunit is outlined below.

Caseins: Gene sequences that encode for bovine α-SI-casein (P02662) and bovine ß-casein (P02666) were derived from Uniprot and modified to include the following C-terminus 60 bp sequence (SEQ ID No. 6):

(AGTTCG) (GATTACAAAGATGACGACGATAAG) (CATCATCACCATCACCAC) (CATGATGA GTTG). The first sequence encodes for a two-serine spacer that functions to provide enough space for each casein protein to properly fold without steric hindrance or interference from other tags. The second and third sequences encode for two affinity purification tags (flag-tag and 6-Histidine tag) that are commonly used for protein identification and purification. The fourth sequence encodes for an HDEL target peptide that functions to retain soluble casein proteins in the endoplasmic reticulum (ER). Additionally, an N-terminus signal peptide, gmGlycininl (GYI, P04776), was added to all three caseins in order to target them towards the ER and vacuoles. Recombinant casein sequences were expressed under the constitutive AtuMas promoter and 5′ untranslated region (UTR) (https://www.ncbi.nlm.nih.gov/pmc/arti cl es/PMC287101/).

Gene sequence encoding for bovine κ-casein (P02668) was derived from Uniprot and modified to include the following 57 bp sequence to increase the hydrophilicity of the C-terminus region, following the sequence (SEQ ID No. 7):

(SEQ ID NO. 8)
TCGGATTACAAAGATGACGACGATAAGCATCATCACCATCACCACCAT
GATGAGTTG, corresponding to amino acid sequence
SDYKDDDDKHHHHHHHDEL

Additionally, an N-terminus signal peptide, gmGlycininl (GYI, P04776), was added to all three caseins in order to target them towards the ER and vacuoles. Recombinant casein sequences were expressed under the constitutive AtuMas promoter and 5′ untranslated region (UTR).

FAM20C kinase: The gene sequence of for the FAM20C kinase (F1 MXQ3) was modified to include the N-terminus At5g67360 signal peptide sequence and a C-terminus HDEL target peptide sequence (CACGATGAGTTA). The recombinant FAM20C kinase was expressed under the 35S short promoter.

Green fluorescent protein (GFP): Cytosolic GFP (P42212) was expressed under the 35S short promoter and used as a visual reporter for detecting the expression of pMOZ1066 in transient soybean agrobacterium transformations.

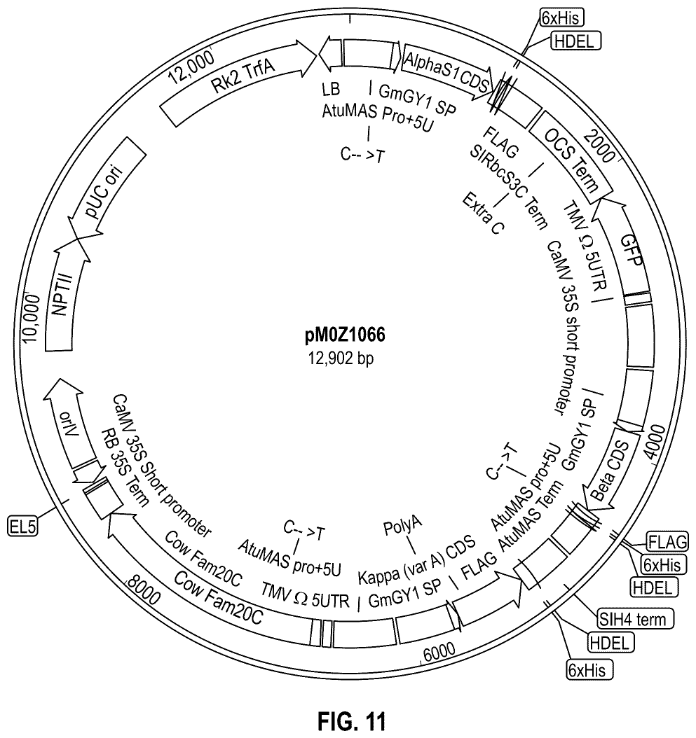

E. coli transformation: pMOZ1066 (FIG. 11, SEQ ID NO. 9) was transformed into Lucigen Ecloni I OG bacteria using the following chemical transformation protocol: First, Lucigen Ecloni 10G E. coli thermo-competent cells were thawed on ice for approximately 5 minutes. Then, competent cells were spiked with 1 pg-100 ng of plasmid DNA and left to incubate on ice for 10 minutes. Once complete, the plasmid-bacterial mixture was heat shocked at 42° C. for 90 seconds and immediately replaced on ice for 5 minutes. Afterwards, transformed cells were mixed with 10 μl of liquid broth (LB) and cultured in a 37° C. shaker set to 225 rpm for 45 minutes. Following incubation, cultured bacteria was plated on LB agar plates with the appropriate antibiotic for selection (1:1000 concentration) and left to further incubate overnight in a 37° C. growth chamber.

High throughput blue-white selection: Following overnight incubation, plates were checked for bacterial colony growth. To increase recombinant bacteria selection, a MoClo compatible blue-white selection system was used. In brief, blue-white selection plasmid vectors carry the “lacZ” operon sequence within their multiple cloning site. In the absence of recombinant DNA, the lacZ operon will enable a biochemical reaction that turns the colony blue. Whereas, when recombination occurs, lacZ operon activity will be disrupted leaving the colony to be white. For each cloning reaction, at least two white colonies were chosen for further verification.

Plasmid selection and verification: Picked colonies were placed in 5 mLs of LB plus their respective selection antibiotic (1:1000 concentration) and cultured overnight in a 37° C. shaker (225 rpm). Once cloudy, plasmids were purified out of bacteria using the NucleoSpin miniprep kit (Takara bio inc.). Then, each plasmid was digested using the appropriate restriction enzymes in order to confirm the presence of a DNA insert and sent for sanger and nanopore sequencing to confirm sequence correctness. Colonies containing the correct plasmids were made into frozen 20% glycerol stocks.

Electroporation into agrobacterium: The completed pMOZ1066 plasmid was transformed into EHA105 electrocompetent agrobacterium cells. To achieve this, 30 ng of pMOZ1066 purified plasmid was mixed into ice-thawed electrocompetent cells and swiftly transferred into a pre-chilled 0.2 cm Gene pulser cuvette. The cuvette was then loaded into an electroporation chamber and given an electric pulse of 2.5 kV. The resulting transformed cells were mixed with 500 μl of LB and left to culture in a 28° C. shaking incubator (120 rpm) for 2-4 hours. Afterwards, cultured cells were plated onto LB agar plates containing the appropriate antibiotic selection media and placed in a 28° C. incubator for two days. Once colonies formed, a minimum of three were picked for further verification via purification, digestion, and sequencing.

Transient agrobacterium transformation into zygotic soy embryos: Zygotic soybeans were transiently transformed using agrobacterium. Specifically, a 5 mL starter culture of pMOZ1066 agrobacterium was started from either a glycerol stock or bacterial colony and incubated for two days in a 28° C. shaker (120 rpm). Once cloudy, the 5 mL starter culture was used to inoculate a larger 200 mL overnight culture.

Bacteria preparation: Large pMOZ1066 agrobacterium cultures were spundown in a large centrifuge at 3400g for 10 minutes. The supernatant was then removed and the remaining agrobacterium pellet was resuspended in 30 mL of LCCM. Post resuspension, the agrobacterium was centrifuged at 3400g for 6 minutes, and then subjected to one more round of supernatant removal, pellet resuspension and centrifugation. After the final spindown, the remaining supernatant was removed and the bacterial pellet was resuspended in 10 mL of LCCM. An OD600 measurement was then taken and the agrobacterium solution was diluted to a final concentration of 1.4 OD600. The resulting agrobacterium solution was spiked with fresh acetosyringone (100 μM final concentration) and subsequently incubated at room temperature while shaking (120 rpm) for 1-2 hours.

Seed sterilization and preparation: During the agrobacterium incubation period, pods were picked from soy plants containing 8-10 mm embryos (˜8 weeks old) and sterilized by the following method: 70% ethanol bath for 30 seconds, 10% bleach bath for 10 minutes, three sequential sterilized deionized water bath for 5 minutes each. After sterilization, seeds were aseptically removed from their pods, dissected from their seed coats, and split in half.

Explant inoculation: Once agrobacterium cultures finished incubating, silwet-77 (0.03% v/v) was added and mixed until dissolved. Then, 30 cotyledon halves (15 explants) were placed in the agrobacterium culture, and sonicated for 20 seconds at a 20% amplitude with 5s/l/s on/off pulse cycles. After sonication, explants were vacuum infiltrated for 5 minutes and left to incubate for two hours on a room temperature rotator.

Plating explants: Explants were removed from bacterial culture and placed flat down (adaxial side down) on SCCM plates with a layer of sterile filter paper. Plates were then wrapped with micropore tape and incubated for 3 days in a dark 24° C. chamber.

Washing explants: After plants incubated in the dark for 3 days, they were washed three times for five minutes each with sterile water containing Rif, Carb+Cef. Then, they were replated on SCCM plates containing filter paper and left to incubate in a dark 24° C. chamber for another 7-9 days.

Method of detecting formation of in-vivo micelles by FLAG-tagged casein genes in soybean embryos:
1. Transformation: Transform soybean embryos with a plasmid containing casein genes fused to FLAG-tags (experimental) or a plasmid without casein genes (control).
2. Tissue fixation: Fix the transformed soybean embryos in a fixative solution.
3. Resin embedding: Embed the fixed tissues in LR White resin.
4. Ultramicrotomy: Cut thin sections (approximately 60 nm) from the embedded tissues using an ultramicrotome.
5. Grid mounting: Mount the sections on a carbon grid stage.
6. Antibody staining: Stain the grids with an anti-FLAG primary antibody to detect the FLAG-tagged casein proteins.
7. Secondary antibody staining: Stain the grids with a gold-nanoparticle secondary antibody that binds to the primary antibody.
8. Imaging: Image the stained grids using a ThermoFisher TALOS transmission electron microscope.

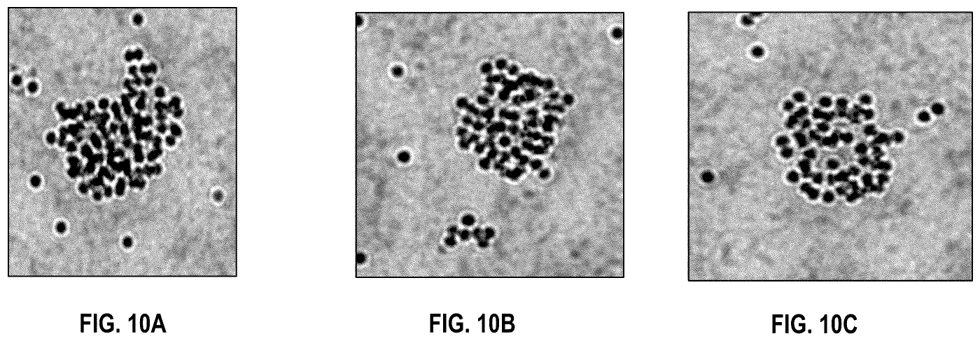

Result: The experimental samples showed the presence of gold-nanoparticle labeling indicating the presence of FLAG-tagged casein proteins, whereas the control samples showed no labeling. In addition, the flag-tagged casein proteins formed aggregates between 50 and 200 nm evidencing the formation of casein micelles (FIG. 10)

TABLE 1
Sequence IDs in Example 4

| SEQ ID | Description | Sequence |
|---|---|---|
| SEQ ID | bovine α- | RPKHPIKHQGLPQEVLNENLLRFFVAPFPEVFGKEKVNELSK |
| NO. 1 | S1-casein | DIGSESTEDQAMEDIKQNIEAESISSSEEIVPNSVEQKHIQKED |
| | | VPSERYLGYLEQLLRLKKYKVPQLEIVPNSAEERLHSN/KEGI |
| | | HAQQKEPMIGVNQELAYFYPELFRQFYQLDAYPSGAWYYVP |
| | | LGTQYTDAPSFSDIPNPIGSENSEKTTMPLW |
| SEQ ID | green | MVSKGEELFTGVVPILVELDGDVNGHKFSVSGEGEGDATYG |
| NO. 2 | fluorescent | KLTLKFICTTGKLPVPWPTLVTTFSYGVQCFSRYPDHVKQHD |
| | protein | FFKSAMPEGYVQERTIFFKDDGNYKTRAEVKFEGDTLVNRIE |
| | | LKGIDFKEDGNILGHKLEYNYNSHNVYIMADKQKNGIKVNF |
| | | KIRHNIEDGSVQLADHYQQNTPIGDGPVLLPDNHYLSTQSAL |
| | | SKDPNEKRDHVIVLLEFVTAAGITHGMDELYK* |
| SEQ ID | bovine β- | RELEELNVPGEIVESLSSSEESITRINKKIEKFQSEEQQQTEDEL |
| NO. 3 | casein | QDKIHPFAQTQSLVYPFPGPIPNSLPQNIPPLTQTPVVVPPFLQ |
| | | PEVMGVSKVKEAMAPKHKEMPFPKYPVEPFTESQSLTLTDV |
| | | ENLFHNLPLLQSWMHQPHQPLPPTVMFPPQSVLSLSQSKVLP |
| | | VPQKAVPYPQRDMPIQAFLLYQEPVLGPVRGPFPII |
| SEQ ID | bovine κ- | QEQNQEQPIRCEKDERFFSDKIAKYIPIQYVLSRYPSYGLNYY |
| NO. 4 | casein | QQKPVALINNQFLPYPYYAKPAAVRSPAQILQWQVLSNTVPAK |
| | | SCQAQPTTMARHPHPHLSFMAIPPKKNQDKTEIPTINTIAS |
| | | GEPTSTPTTEAVESTVATLEDSPEVIESPPEINTVQVTSTA |
| SEQ ID | bovine | MSSSFLSSTAFFLLLCLGFCHVSSLLPKLERSAARPSGEPGCSC |
| NO. 5 | FAM20C | AQPAAEAAAPGWAQARGFWGGELEAAASAAGDAGWPNKH |
| | kinase | TLRILQDFSSDPSSNLTSHSLEKLPPAAEAAEGAPPGQDPGVR |
| | | RPPDPAHRPLPRDPGPRGPVLPPGLSGDGSLLTRLFQHPLYQV |
| | | PIPPLTEGDVLFNVNSDRFNPKAATAENPDWPHEGPEDEFLP |
| | | TGEAAVDSYPNWLKFHIGINRYELYSRHNPAVGALLQDLGT |
| | | QKITSVAMKSGGTQLKLnv1TFQNYGQALFKPMKQTREQETP |
| | | PDFFYFSDYERHNAEIAAFHLDRILDFRRVPPVAGRLVNMTK |
| | | EIRDVTRDKKLWRTFFISPANNVCFYGECSYYCSTEHALCGK |
| | | PDQIEGSLAAFLPDLALAKRKTWRNPWRRSYHKRKKAEWE |
| | | VDPDYCEEVRQTPPYDSSHRLLDVMDMTIFDFLMGNMDRH |
| | | HYETFEKFGNETFIIHLDNGRGFGKHSHDELSILVPLQQCCRI |
| | | RRSTYLRLQLLAQEEHRLSLLMAEALRADRVAPVLFQPHLE |
| | | ALDRRLRIVLRAVGDCVEKDGLHSVVEDDLGPEHRAAAGR |
| | | HDEL* |
| SEQ ID | C-terminus | AGTTCGGATTACAAAGATGACGACGATAAGCATCATCACC |
| NO. 6 | 60 bp | ATCACCACCATGATGAGTTG |
| | sequence | |
| SEQ ID | C-terminus | TCGGATTACAAAGATGACGACGATAAGCATCATCACCATC |
| NO. 7 | sequence | ACCACCATGATGAGTTG |
| SEQ ID | C-terminus | SDYKDDDDKHHHHHHHDEL |
| NO. 8 | amino acid | |
| | sequence | |
| SEQ ID | pMOZ1066 | gtgccgaattcggatccGGAGATTTTTCAAATCAGTGCGCAAGACG |
| NO. 9 | | TGACGTAAGTATCCGAGTCAGTTTTTATTTTTCTACTAATT |
| | | TGGTCGTTTATTTCGGCGTGTAGGACATGGCAACCGGGCC |
| | | TGAATTTCGCGGGTATTCTGTTTCTATTCCAACTTTTTCTTG |
| | | ATCCGCAGCCATTAACGACTTTTGAATAGATACGCTGACA |
| | | CGCCAAGCCTCGCTAGTTAAAAGTGTACCAAACAACGCTT |
| | | TACAGCAAGAACGGAATGCGCGTGACGCTCGCGGTGACG |
| | | CCATTTCGCCTTTTCAGAAATGGATAAATAGCCTTGCTTCC |
| | | TATTATATCTTCCCAAATTACCAATACATTACACTAGCATC |
| | | TGAATTTCATAACCAATCTCGATACACCAAATCGCCATGG |
| | | CCAAGCTAGTTTTTTCCCTTTGTTTTCTGCTTTTCAGTGGCT |
| | | GCTGCTTCGCaATGCGTCCGAAGCACCCAATCAAGCATCA |
| | | AGGGCTTCCCCAAGAGGTACTGAACGAAAATCTACTCAGA |
| | | TTCTTCGTGGCTCCATTTCCTGAGGTATTTGGGAAGGAAA |
| | | AGGTGAATGAGCTGTCTAAAGACATAGGCTCTGAATCAAC |
| | | TGAGGACCAAGCTATGGAGGATATAAAGCAAATGGAGGC |
| | | CGAATCCATATCCTCAAGCGAGGAAATCGTTCCGAATTCC |
| | | GTCGAACAGAAGCATATTCAAAAGGAGGATGTGCCTTCTG |
| | | AGCGATACCTTGGATATCTTGAACAGTTGCTGAGACTAAA |
| | | GAAATACAAGGTCCCCCAACTAGAAATTGTTCCTAACTCA |
| | | GCCGAAGAGCGATTACACTCTATGAAAGAGGGGATTCAT |
| | | GCTCAACAGAAGGAGCCTATGATCGGTGTGAATCAAGAA |
| | | CTGGCATACTTCTATCCCGAACTGTTTCGACAATTTTATCA |
| | | ATTAGATGCGTATCCATCCGGGGCGTGGTATTATGTGCCT |
| | | CTTGGGACACAGTATACGGATGCGCCGAGTTTTTCAGACA |
| | | TCCCAAACCCAATAGGCAGTGAGAATTCAGAAAAGACGA |
| | | CAATGCCTCTTTGGAGTTCGGATTACAAAGATGACGACGA |
| | | TAAGCATCATCACCATCACCACCATGATGAGTTGTAGGCT |
| | | TATATGTCAACAGTGAGAAACTGTTCGCATTTTCCGTTTTG |
| | | CTTCTTTCTTTCTATTCAATGTATGTTGTTGGATTCCAGTTG |
| | | AATTTATTATGAGAACTAATAATAATAGTAATAATCATTT |
| | | GTTTCTTTACTAATTTGCATTTTCACATATGATTTCTGGTG |
| | | CATATCATAATTTTCATTCCACCAATATTAATTTCCCCCAT |
| | | TCAAGTTACTTATGAAATAGAAATCCTCTTCTCCGACTACT |
| | | TTATTTGTCCGAAAGTCTTGTGGCTGCTATATAAcgctgcaaga |
| | | attcaagcttagcgGTCCTGCTGAGCCTCGACATGTTGTCGCAAAA |
| | | TTCGCCCTGGACCCGCCCAACGATTTGTCGTCACTGTCAA |
| | | GGTTTGACCTGCACTTCATTTGGGGCCCACATACACCAAA |
| | | AAAATGCTGCATAATTCTCGGGGCAGCAAGTCGGTTACCC |
| | | GGCCGCCGTGCTGGACCGGGTTGAATGGTGCCCGTAACTT |
| | | TCGGTAGAGCGGACGGCCAATACTCAACTTCAAGGAATCT |
| | | CACCCATGCGCGCCGGCGGGGAACCGGAGTTCCCTTCAGT |
| | | GAGCGTTATTAGTTCGCCGCTCGGTGTGTCGTAGATACTA |
| | | GCCCCTGGGGCACTTTTGAAATTTGAATAAGATTTATGTA |
| | | ATCAGTCTTTTAGGTTTGACCGGTTCTGCCGCTTTTTTTAA |
| | | AATTGGATTTGTAATAATAAAACGCAATTGTTTGTTATTGT |
| | | GGCGCTCTATCATAGATGTCGCTATAAACCTATTCAGCAC |
| | | AATATATTGTTTTCATTTTAATATTGTACATATAAGTAGTA |
| | | GGGTACAATCAGTAAATTGAACGGAGAATATTATTCATAA |
| | | AAATACGATAGTAACGGGTGATATATTCATTAGAATGAAC |
| | | CGAAACCGGCGGTAAGGATCTGAGCTACACATGCTCAGGT |
| | | TTTTTACAACGTGCACAACAGAATTGAAAGCAAATATCAT |
| | | GCGATCATAGGCTTCTCGCATATCTCATTAAAGCAGGACA |
| | | AGCTTACTTGTACAGCTCGTCCATGCCGTGAGTGATCCCG |
| | | GCGGCGGTCACGAACTCCAGCAGGACCATGTGATCGCGCT |
| | | TCTCGTTGGGGTCTTTGCTCAGGGCGGACTGGGTGCTCAG |
| | | GTAGTGGTTGTCGGGCAGCAGCACGGGGCCGTCGCCGATG |
| | | GGGGTGTTCTGCTGGTAGTGGTCGGCGAGCTGCACGCTGC |
| | | CGTCCTCGATGTTGTGGCGGATCTTGAAGTTCACCTTGATG |
| | | CCGTTCTTCTGCTTGTCGGCCATGATATAGACGTTGTGGCT |
| | | GTTGTAGTTGTACTCCAGCTTGTGCCCCAGGATGTTGCCGT |
| | | CCTCCTTGAAGTCGATGCCCTTCAGCTCGATGCGGTTCACC |
| | | AGGGTGTCGCCCTCGAACTTCACCTCGGCGCGGGTCTTGT |
| | | AGTTGCCGTCGTCCTTGAAGAAGATGGTGCGCTCCTGGAC |
| | | GTAGCCTTCGGGCATGGCGGACTTGAAGAAGTCGTGCTGC |
| | | TTCATGTGGTCGGGGTAGCGGCTGAAGCACTGCACGCCGT |
| | | AGCTGAAGGTGGTCACGAGGGTGGGCCAGGGCACGGGCA |
| | | GCTTGCCGGTGGTGCAGATGAACTTCAGGGTCAGCTTGCC |
| | | GTAGGTGGCATCGCCCTCGCCCTCGCCGGACACGCTGAAC |
| | | TTGTGGCCGTTTACGTCGCCGTCCAGCTCGACCAGGATGG |
| | | GCACCACCCCGGTGAACAGCTCCTCGCCCTTGCTCACCAT |
| | | TGTATCGATAATTGTAAATGTAATTGTAATGTTGTTTGTTG |
| | | TTTGTTGTTGTTGGTAATTGTTGTAAAAATGAGCTCTTATA |
| | | CTCGAGCGTGTCCTCTCCAAATGAAATGAACTTCCTTATAT |
| | | AGAGGAAGGGTCTTGCGAAGGATAGTGGGATTGTGCGTC |
| | | ATCCCTTACGTCAGTGGAGATGTCACATCAATCCACTTGC |
| | | TTTGTAGACGTGGTTGGAACCTCTTCTTTTTCCACGATGCT |
| | | CCTCGTGGGTGGGGGTCCATCTTTGGGACCACTGTCGGCA |
| | | GAGAGATCTTGAATGATAGCCTTTCCTTTATCGCAATGAT |
| | | GGCATTTGTAGGAGCCACCTTCCTTTTCTACTGTCCTTTCG |
| | | ATGAAGTGACAGATAGCTGGGCAATGGAATCCGAGGAGG |
| | | TTTCCCGAAATTATCCTTTGTTGAAAAGTCTCAATAGCCCT |
| | | TTGATCTTCTGAGACTGTATCTTTGACATTTTTGGAGTAGA |
| | | CCAGAGTGTCGTGCTCCACCATGTTGACCTCCactagaattegagc |
| | | tcGGAGATTTTTCAAATCAGTGCGCAAGACGTGACGTAAGT |
| | | ATCCGAGTCAGTTTTTATTTTTCTACTAATTTGGTCGTTTAT |
| | | TTCGGCGTGTAGGACATGGCAACCGGGCCTGAATTTCGCG |
| | | GGTATTCTGTTTCTATTCCAACTTTTTCTTGATCCGCAGCC |
| | | ATTAACGACTTTTGAATAGATACGCTGACACGCCAAGCCT |
| | | CGCTAGTTAAAAGTGTACCAAACAACGCTTTACAGCAAGA |
| | | ACGGAATGCGCGTGACGCTCGCGGTGACGCCATTTCGCCT |
| | | TTTCAGAAATGGATAAATAGCCTTGCTTCCTATTATATCTT |
| | | CCCAAATTACCAATACATTACACTAGCATCTGAATTTCAT |
| | | AACCAATCTCGATACACCAAATCGCCATGGCCAAGCTAGT |
| | | TTTTTCCCTTTGTTTTCTGCTTTTCAGTGGCTGCTGCTTCGC |
| | | aATGAGAGAACTTGAAGAATTGAATGTTCCAGGAGAGATT |
| | | GTTGAAAGTCTCTCCTCTTCTGAGGAAAGCATCACAAGAA |
| | | TCAACAAGAAAATTGAGAAGTTTCAGAGTGAAGAGCAAC |
| | | AACAGACTGAAGATGAATTACAAGATAAGATTCATCCTTT |
| | | TGCTCAAACACAGTCACTTGTGTATCCATTCCCTGGACCA |
| | | ATTCCAAATTCTTTACCACAAAACATTCCTCCTCTGACTCA |
| | | AACTCCTGTCGTTGTTCCTCCGTTCTTGCAACCAGAAGTTA |
| | | TGGGAGTTTCAAAGGTTAAAGAAGCAATGGCTCCAAAGC |
| | | ATAAAGAGATGCCATTCCCAAAATACCCTGTGGAGCCTTT |
| | | CACAGAATCTCAAAGCTTGACTCTCACTGATGTTGAGAAT |
| | | CTTCATTTGCCTCTTCCATTGCTTCAATCATGGATGCATCA |
| | | ACCTCATCAGCCTTTGCCACCAACAGTGATGTTTCCACCTC |
| | | AATCTGTTCTCTCTCTTTCTCAGTCTAAAGTTCTTCCGGTTC |
| | | CGCAGAAAGCTGTGCCTTATCCTCAGAGAGATATGCCTAT |
| | | TCAAGCTTTTCTTCTCTACCAAGAACCAGTTTTGGGTCCTG |
| | | TTCGTGGTCCATTTCCCATCATAGTTTCGGATTACAAAGAT |
| | | GACGACGATAAGCATCATCACCATCACCACCATGATGAGT |
| | | TGTAGGCTTGGACTCCCATGTTGGCAAAGGCAACCAAACA |
| | | AACAATGAATGATCCGCTCCTGCATATGGGGCGGTTTGAG |
| | | TATTTCAACTGCCATTTGGGCTGAATTGTAGACATGCTCCT |
| | | GTCAGAAATTCCGTGATCTTACTCAATATTCAGTAATCTCG |
| | | GCCAATATCCTAAATGTGCGTGGCTTTATCTGTCTTTGTAT |
| | | TGTTTCATCAATTCATGTAACGTTTGCTTTTCTTATGAATTT |
| | | TCAAATAAATTATCcgctttacgaattcccatggagcgctattactcaaattctcatta |
| | | atttcaagaattttcttaatttcacagtatacaatttaaattcagaagaattgcaataggatgaatca |
| | | attaagaaaaattaatcaacagctaaaagacgaaccaatgtcatcactagaaactgatatctattcatt |
| | | gatactgtaatgagaaattacaagatctaagccataagaacagcactacagatatccaacacaga |
| | | catacgcaattgtaaactaaatccctacatttaccaccatatcaccaacataaaagcCTACAA |
| | | CTCATCATGGTGGTGATGGTGATGATGCTTATCGTCGTCAT |
| | | CTTTGTAATCCGAAACAGCCGTTGAGGTGACCTGTACCGT |
| | | GTTTATCTCCGGGGGTGATTCAATCACCTCTGGGGAATCTT |
| | | CAAGTGTGGCGACCGTGCTCTCCACCGCTTCAGTAGTCGG |
| | | TGTTGAAGTTGGCTCACCAGAAGCGATAGTATTTATGGTC |
| | | GGGATCTCTGTCTTGTCTTGGTTCTTTTTGGGAGGAATAGC |
| | | CATAAAGGAAAGGTGCGGATGCGGGTGTCTAGCCATGGT |
| | | GGTGGGTTGCGCCTGGCAAGACTTCGCCGGGACAGTGTTG |
| | | GATAAAACCTGCCACTGCAATATCTGAGCAGGAGAACGG |
| | | ACGGCGGCCGGCTTAGCATAGTAGGGATATGGGAGAAAC |
| | | TGGTTGTTTATCAAGGCAACTGGCTTCTGTTGATAGTAATT |
| | | AAGACCGTAGCTCGGATATCGACTTAAAACGTACTGGATT |
| | | GGGATGTACTTTGCAATTTTATCACTAAAAAATCTCTCGTC |
| | | TTTTTCACAACGAATAGGTTGCTCTTGGTTTTGCTCCTGCA |
| | | TtGCGAAGCAGCAGCCACTGAAAAGCAGAAAACAAAGGG |
| | | AAAAAACTAGCTTGGCCATGGCGATTTGGTGTATCGAGAT |
| | | TGGTTATGAAATTCAGATGCTAGTGTAATGTATTGGTAAT |
| | | TTGGGAAGATATAATAGGAAGCAAGGCTATTTATCCATTT |
| | | CTGAAAAGGCGAAATGGCGTCACCGCGAGCGTCACGCGC |
| | | ATTCCGTTCTTGCTGTAAAGCGTTGTTTGGTACACTTTTAA |
| | | CTAGCGAGGCTTGGCGTGTCAGCGTATCTATTCAAAAGTC |
| | | GTTAATGGCTGCGGATCAAGAAAAAGTTGGAATAGAAAC |
| | | AGAATACCCGCGAAATTCAGGCCCGGTTGCCATGTCCTAC |
| | | ACGCCGAAATAAACGACCAAATTAGTAGAAAAATAAAAA |
| | | CTGACTCGGATACTTACGTCACGTCTTGCGCACTGATTTGA |
| | | AAAATCTCCcagagaattcgcatgcGGAGGTCAACATGGTGGAGC |
| | | ACGACACTCTGGTCTACTCCAAAAATGTCAAAGATACAGT |
| | | CTCAGAAGATCAAAGGGCTATTGAGACTTTTCAACAAAGG |
| | | ATAATTTCGGGAAACCTCCTCGGATTCCATTGCCCAGCTA |
| | | TCTGTCACTTCATCGAAAGGACAGTAGAAAAGGAAGGTG |
| | | GCTCCTACAAATGCCATCATTGCGATAAAGGAAAGGCTAT |
| | | CATTCAAGATCTCTCTGCCGACAGTGGTCCCAAAGATGGA |
| | | CCCCCACCCACGAGGAGCATCGTGGAAAAAGAAGAGGTT |
| | | CCAACCACGTCTACAAAGCAAGTGGATTGATGTGACATCT |
| | | CCACTGACGTAAGGGATGACGCACAATCCCACTATCCTTC |
| | | GCAAGACCCTTCCTCTATATAAGGAAGTTCATTTCATTTGG |
| | | AGAGGACACGCTCGAGTATAAGAGCTCATTTTTACAACAAT |
| | | TAC C ACAACAACAAAC AAC AAAC AT TAC AAT TAC |
| | | ATTTACAATTATCGATACaATGAGCAGCAGTTTCTTGAGCT |
| | | CAACCGCTTTCTTTCTATTACTCTGTCTGGGGTTCTGTCAC |
| | | GTGAGCAGTCTCCTACCCAAATTAGAACGATCTGCTGCAC |
| | | GTCCGAGCGGCGAACCTGGCTGTAGCTGCGCACAGCCAGC |
| | | TGCCGAAGCTGCCGCGCCTGGATGGGCTCAAGCTAGGGGT |
| | | CATCCCGGTGGAGAACTTGAAGCGGCCGCTAGCGCCGCCG |
| | | GGGATGCAGGCTGGCCAAATAAGCACACTCTGAGGATTCT |
| | | GCAAGACTTCAGTTCCGACCCCAGTTCCAACCTAACGAGC |
| | | CACTCACTGGAAAAGCTGCCTCCGGCTGCCGAAGCTGCGG |
| | | AAGGTGCACCGCCAGGCCAAGATCCAGGAGTTCGTAGAC |
| | | CTCCCGACCCAGCGCATAGGCCACTCCCGCGAGATCCGGG |
| | | TCCTAGAGGCCCTGTCTTGCCCCCAGGTCTTAGCGGAGAC |
| | | GGGTCCTTACTTACGCGTCTTTTCCAACACCCGCTATACCA |
| | | GGTGCCCATACCGCCCCTAACAGAAGGCGATGTTCTCTTT |
| | | AATGTCAATAGCGATATAAGATTCAACCCCAAAGCTGCAA |
| | | CCGCCGAGAACCCAGATTGGCCACACGAGGGGCCGGAAG |
| | | ATGAGTTTTTACCTACTGGTGAAGCGGCAGTTGACTCTTA |
| | | CCCGAATTGGCTGAAGTTTCATATTGGGATCAATAGATAC |
| | | GAGCTTTACAGCCGACATAATCCGGCCGTGGGAGCGCTCT |
| | | TACAAGACCTCGGGACGCAAAAGATTACTTCTGTCGCTAT |
| | | GAAATCTGGCGGGACACAGCTCAAACTTATTATGACTTTC |
| | | CAGAATTATGGCCAAGCTCTGTTCAAGCCGATGAAGCAGA |
| | | CTAGAGAGCAGGAGACACCCCCTGACTTCTTCTACTTCAG |
| | | CGACTATGAAAGGCATAATGCAGAAATTGCGGCATTCCAC |
| | | CTTGATAGGATCTTAGACTTCCGAAGGGTACCACCGGTAG |
| | | CAGGTAGACTAGTCAATATGACTAAAGAGATTAGAGATGT |
| | | CACTCGTGACAAGAAACTATGGCGTACATTCTTTATAAGC |
| | | CCTGCTAACAATGTATGCTTTTATGGCGAATGTTCTTACTA |
| | | TTGCTCTACAGAACATGCACTGTGTGGAAAACCCGACCAG |
| | | ATTGAGGGGTCACTAGCCGCATTTCTGCCAGACTTGGCAT |
| | | TGGCCAAGCGTAAGACGTGGCGTAATCCGTGGCGACGTA |
| | | GTTACCACAAGAGAAAGAAGGCGGAGTGGGAAGTAGACC |
| | | CAGACTACTGCGAGGAGGTTAGACAAACACCTCCATATGA |
| | | TTCTAGTCATAGACTGTTGGATGTTATGGACATGACAATTT |
| | | TTGATTTTCTCATGGGGAACATGGATCGTCACCACTACGA |
| | | AACCTTTGAGAAATTCGGCAATGAGACATTCATTATCCAC |
| | | TTAGATAATGGTCGAGGTTTTGGCAAACACAGCCATGACG |
| | | AACTATCTATATTAGTGCCTTTACAACAGTGCTGTAGAAT |
| | | CCGAAGGTCTACCTATTTGAGACTTCAACTGTTGGCCCAA |
| | | GAGGAGCATCGTCTATCACTTTTAATGGCCGAGGCTCTAA |
| | | GGGCTGATCGTGTGGCTCCCGTACTCTTTCAGCCTCACTTA |
| | | GAGGCTTTAGATCGTCGACTTCGTATAGTGCTTCGAGCGG |
| | | TAGGCGATTGCGTGGAGAAAGATGGACTGCACAGTGTTGT |
| | | AGAGGATGATTTGGGGCCTGAGCACAGGGCGGCCGCGGG |
| | | ACGTCACGATGAGTTATAAGCTTCTCTAGCTAGAGTCGAT |
| | | CGACAAGCTCGAGTTTCTCCATAATAATGTGTGAGTAGTT |
| | | CCCAGATAAGGGAATTAGGGTTCCTATAGGGTTTCGCTCA |
| | | TGTGTTGAGCATATAAGAAACCCTTAGTATGTATTTGTATT |
| | | TGTAAAATACTTCTATCAATAAAATTTCTAATTCCTAAAAC |
| | | CAAAATCCAGTACTAAAATCCAGATcgcttgtggaggatgcacatgtga |
| | | ccgagggacacgaagtgatccgtttaaactatcagtgtttgacaggatatattggcgggtaaacct |
| | | aagagaaaagagcgtttattagaataatcggatatttaaaagggcgtgaaaaggtttatccgttcgt |
| | | ccatttgtatgtgccagccgcctttgcgacgctcaccgggctggttgccctcgccgctgggctgg |
| | | cggccgtctatggccctgcaaacgcgccagaaacgccgtcgaagccgtgtgcgagacaccgc |
| | | ggccgccggcgttgtggatacctcgcggaaaacttggccctcactgacagatgaggggcggac |
| | | gttgacacttgaggggccgactcacccggcgcggcgttgacagatgaggggcaggctcgattt |
| | | cggccggcgacgtggagctggccagcctcgcaaatcggcgaaaacgcctgattttacgcgagt |
| | | ttcccacagatgatgtggacaagcctggggataagtgccctgcggtattgacacttgaggggcg |
| | | cgactactgacagatgaggggcgcgatccttgacacttgaggggcagagtgctgacagatgag |
| | | gggcgcacctattgacatttgaggggctgtccacaggcagaaaatccagcatttgcaagggtttc |
| | | cgcccgtttttcggccaccgctaacctgtcttttaacctgcttttaaaccaatatttataaaccttgt |
| | | ttttaaccagggctgcgccctgtgcgcgtgaccgcgcacgccgaaggggggtgcccccccttctcg |
| | | aaccctcccggcccgctaacgcgggcctcccatccccccaggggctgcgcccctcggcegcg |
| | | aacggcctcaccccaaaaatggcagcgctggccaattcccgagtgcgcggaacccctatttgttt |
| | | atttttctaaatacattcaaatatgtatccgctcatgagacaataaccctgataaatgcttcaataat |
| | | attgaaaaaggaagagtatggctaaaatgagaatatcaccggaattgaaaaaactgatcgaaaaat |
| | | accgctgcgtaaaagatacggaaggaatgtctcctgctaaggtatataagctggtgggagaaaat |
| | | gaaaacctatatttaaaaatgacggacagccggtataaagggaccacctatgatgtggaacggg |
| | | aaaaggacatgatgctatggctggaaggaaagctgcctgttccaaaggtcctgcactttgaacgg |
| | | catgatggctggagcaatctgctcatgagtgaggccgatggcgtcctttgctcggaagagtatga |
| | | agatgaacaaagccctgaaaagattatcgagctgtatgcggagtgcatcaggctctttcactccat |
| | | cgacatatcggattgtccctatacgaatagcttagacagccgcttagccgaattggattacttactg |
| | | aataacgatctggccgatgtggattgcgaaaactgggaagaggacactccatttaaagatccgc |
| | | gcgagctgtatgattttttaaagacggaaaagcccgaagaggaacttgtcttttcccacggcgacc |
| | | tgggagacagcaacatctttgtgaaagatggcaaagtaagtggctttattgatcttgggagaagc |
| | | ggcagggcggacaagtggtatgacattgccttctgcgtccggtcgctcagggaggatatcggg |
| | | gaagaacagtatgtcgagctattttttgacttactggggatcaagcctgattgggagaaaataaaat |
| | | attatattttactggatgaattgttttagctgtcagaccaagtttactcatatatactttagattgat |
| | | ttaaaacttcatttttaatttaaaaggatctaggtgaagatcctttttgataatctcatgaccaaaat |
| | | cccttaacgtgagttttcgttccactgagcgtcagaccccgtagaaaagatcaaaggatcttcttgag |
| | | atcctttttttctgcgcgtaatctgctgcttgcaaacaaaaaaaccaccgctaccagcggtggtttgt |
| | | ttgccggatcaagagctaccaactctttttccgaaggtaactggcttcagcagagcgcagataccaaa |
| | | tactgtccttctagtgtagccgtagttaggccaccacttcaagaactctgtagcaccgcctacatacct |
| | | cgctctgctaatcctgttaccagtggctgctgccagtggcgataagtcgtgtcttaccgggttgga |
| | | ctcaagacgatagttaccggataaggcgcagcggtcgggctgaacggggggttcgtgcacaca |
| | | gcccagcttggagcgaacgacctacaccgaactgagatacctacagcgtgagctatgagaaag |
| | | cgccacgcttcccgaagggagaaaggcggacaggtatccggtaagcggcagggtcggaaca |
| | | ggagagcgcacgagggagcttccagggggaaacgcctggtatctttatagtcctgtcgggtttc |
| | | gccacctctgacttgagcgtcgatttttgtgatgctcgtcaggggggcggagcctatggaaaaac |
| | | gccagcaacgcggcctttttacggttcctggcagatcctagatgtggcgcaacgatgccggcga |
| | | caagcaggagcgcaccgacttcttccgcatcaagtgttttggctctcaggccgaggcccacggc |
| | | aagtatttgggcaaggggtcgctggtattcgtgcagggcaagattcggaataccaagtacgaga |
| | | aggacggccagacggtctacgggaccgacttcattgccgataaggtggattatctggacaccaa |
| | | ggcaccaggcgggtcaaatcaggaataagggcacattgccccggcgtgagtcggggcaatcc |
| | | cgcaaggagggtgaatgaatcggacgtttgaccggaaggcatacaggcaagaactgatcgac |
| | | e |
| | | ocoooottttccuccoaouatoccoaaaccategcaaoccocaccgtcatowtococcccoceeee |
| | | gaaaccttccagtccgtcggctcgatggtccagcaagctacggccaagatcgagcgcgacagc |
| | | gtgcaactggctccccctgccctgcccgcgccatcggccgccgtggagcgttcgcgtcgtcttg |
| | | aacaggaggcggcaggtttggcgaagtcgatgaccatcgacacgcgaggaactatgacgacc |
| | | aagaagcgaaaaaccgccggcgaggacctggcaaaacaggtcagcgaggccaagcaggcc |
| | | gcgttgctgaaacacacgaagcagcagatcaaggaaatgcagctttccttgttcgatattgcgcc |
| | | gtggccggacacgatgcgagcgatgccaaacgacacggcccgctctgccctgttcaccacgc |
| | | gcaacaagaaaatcccgcgcgaggcgctgcaaaacaaggtcattttccacgtcaacaaggacg |
| | | tgaagatcacctacaccggcgtcgagctgcgggccgacgatgacgaactggtgtggcagcag |
| | | gtgttggagtacgcgaagcgcacccctatcggcgagccgatcaccttcacgttctacgagctttg |
| | | ccaggacctgggctggtcgatcaatggccggtattacacgaaggccgaggaatgcctgtcgcg |
| | | cctacaggcgacggcgatgggcttcacgtccgaccgcgttgggcacctggaatcggtgtcgct |
| | | gctgcaccgcttccgcgtcctggaccgtggcaagaaaacgtcccgttgccaggtcctgatcgac |
| | | gaggaaatcgtcgtgctgtttgctggcgaccactacacgaaattcatatgggagaagtaccgcaa |
| | | gctgtcgccgacggcccgacggatgttcgactatttcagctcgcaccgggagccgtacccgctc |
| | | aagctggaaaccttccgcctcatgtgcggatcggattccacccgcgtgaagaagtggcgcgag |
| | | caggtcggcgaagcctgcgaagagttgcgaggcagcggcctggtggaacacgcctgggtcaa |
| | | tgatgacctggtgcattgcaaacgctagggccttgtggggtcagttccggctgggggttcagcag |
| | | cccctgctcggatctgttggaccggacagtagtcatggttgatgggctgcctgtatcgagtggtga |
| | | ttttgtgccgagctgccggtcggggagctgttggctggctggtggcaggatatattgtggtgtaaa |
| | | caaattgacgcttagacaacttaataacacattgcggacgtttttaatgtactggggttgaacactct |

### Example 5-In-Vitro Casein Micelle Formation (Milk Making from Individual Casein Proteins)

Dissolve 0.3240 Potassium citrate tribasic in 1.5 ml water to get Tripotassium Citrate

Dissolve 0.383g K2HPO4 in 11 ml water to get Potassium phosphate Dissolve 0470g CaCl2-2H2O in 15 ml of water to get Calcium Chloride.

Extract individual casein proteins from source organism or acquire purified caseins from Sigma Aldrich

Dissolve non-mutant casein proteins with mutant casein protein in water to a concentration of 50 mg/ml to get casein water (or casein containing water).

Add 1 mL of casein containing water to a 5 ml beaker Add a mini stir bar

Place the 5 ml beaker inside of a 600 mL beaker on a hot plate containing approximately 125 ml of water to submerge the 5 mL beaker about a third of the height, using a clamping system (shown at the bottom of the page).

Make sure the 5 ml beaker is not touching the bottom or the sides of the 600 mL beaker.

Place a thermometer in the outer water with the bulb not touching the glass but fully submerged in the water

Set the hotplate around 65C after all of the casein is in solution adjust accordingly to make sure the water temperature stays at 37C (Don't go too high)

Try to get the water temperature to 37C quickly to avoid too much evaporation.

Check the temperature on the thermometer often!

Set the stirring to 1000 RPM

Add the following

20 uL tripotassium citrate

70 uL potassium phosphate is

Wait 4 minutes.

Then every 4 minutes add (12×)

12.5 uL Potassium Phosphate Sol.

25 uL Calcium Chloride Sol.

Let stir with the temperature at 37° C. for 1 hour Turn the heat off

Add 240 uL water

Add 180 uL Heavy Cream

The resulting composition will contain casein in micellar form

### Prophetic Experiment I

Make micelles with all bovine caseins (Sigma Aldrich) using the protocol described herein.

Expected result: OD does not drop substantially over I week.

This is the baseline for comparing caseins.

### Prophetic Experiment 2

Make micelles using only tobacco (or soy) protein, no glycans

Expected result: OD drops over 1 week.

### Prophetic Experiment 3

Make micelles with alpha and beta from plant, bovine kappa

Expected result: OD does not drop

These three experiments show the range of outcomes expected that kappa casein is important for stabilizing micelles and that plant kappa casein without glycans is inferior to bovine kappa casein. The next experiments show how modified plant kappa caseins will improve the stability of micelles made with these modified kappas.

### Prophetic Experiment 4

All plant caseins

Kappa casein having 6 hydrophobic residues (AVILMFYW) swapped for hydrophilic residues (RHKDESTNQ) Expected result: OD doesn't drop after a week

### Prophetic Experiment 5

All plant caseins

Kappa casein having 3 hydrophobic residues selected from AVILMFYW swapped for hydrophilic residues selected from RHKDESTNQ

Pick different residues than experiment 4

Expected result: OD doesn't drop after a week.

### Prophetic Experiment 6

Same as the last two except swap 10 hydrophobic amino acids

Expected result: OD doesn't drop after a week.

### Prophetic Experiment 7

swap non-charged residues (STNQCGPAVILMFYW) for negatively charged residues (DE)

Swap 4 non-charged residues for negatively charged residues

This increases the negative charge of the caseinomacropeptide (e.g., by at least 4) Expected result: OD doesn't drop after a week

### Prophetic Experiment 8

Swap 2 positively charged residues (RK) for 2 negatively charged residues (DE)

This also increases the negative charge of the caseinomacropeptide (e.g., by at least 4) Expected result: OD doesn't drop after a week

### Prophetic Experiment 9

Swap 6 non-charged residues for 6 negatively charged residues

This increases the negative charge of the caseinomacropeptide (e.g., by at least 6) Expected result: OD doesn't drop after a week

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

What is claimed is:

1. A mutant casein protein comprising at least one mutation compared with a wild-type casein protein counterpart, wherein the at least one mutation provides mammalian glycan-related protein functionality.

2. The mutant casein protein in claim 1, wherein the mutant casein protein is a mutated κ-casein.

3. The mutant casein protein in claim 2, wherein the at least one mutation is located on the caseinomacropeptide of the mutated κ-casein.

4. The mutant casein protein in claim 3, wherein the at least one mutation is located at C-terminal caseinomacropeptide of the mutated κ-casein.

5. The mutant casein protein in claim 2, wherein the at least one mutation comprises changing at least one hydrophobic amino acid to a hydrophilic amino acid.

6. The mutant casein protein in claim 2, wherein the at least one mutation comprises changing at least two hydrophobic amino acids to hydrophilic amino acids

7. The mutant casein protein in claim 2, wherein the at least one mutation comprises changing at least three hydrophobic amino acids to hydrophilic amino acids.

8. The mutant casein protein in claim 2, wherein the at least one mutation comprises changing at least six hydrophobic amino acids to hydrophilic amino acids.

9. The mutant casein protein in claim 2, wherein the at least one mutation comprises changing at least ten hydrophobic amino acids to hydrophilic amino acids.

10. The mutant casein protein in any one of claims 5-9, wherein the hydrophobic amino acid is at least one of A, V, I, L, M F, Y, or W, and the hydrophilic amino acid is at least one of R, H, K, D, E, S, T, N, or Q.

11. The mutant casein protein in claim 1, the at least one mutation comprises changing at least one non-charged amino acid to a negatively charged amino acid.

12. The mutant casein protein in claim 11, wherein the at least one mutation comprises changing at least two non-charged amino acids to negatively charged amino acids.

13. The mutant casein protein in claim 11, wherein the at least one mutation comprises changing at least three non-charged amino acids to negatively charged amino acids.

14. The mutant casein protein in claim 11, wherein the at least one mutation comprises changing at least four non-charged amino acids to negatively charged amino acids.

15. The mutant casein protein in claim 11, wherein the non-charged amino acid is at least one of S, T, N, Q, C, G, P, A, V, I, L, M, F, Y or W, and the negatively charged amino acid is D or E.

16. The mutant casein protein in claim 1, at least one mutation comprising changing at least one positively charged amino acid to a negatively charged amino acid.

17. The mutant casein protein in claim 16, wherein the positively charged amino acid is R or K, and the negatively charged amino acid is D or E.

18. The mutant casein protein in any one of claims 11-17, wherein the at least one mutation increases negative charge of caseinomacropeptide.

19. The mutant casein protein in any one of claims 1-18, wherein the mammalian glycan-related protein functionality comprises increased solubility of the mutant casein protein in a liquid.

20. The mutant casein protein in any one of claims 1-18, wherein the mammalian glycan-related protein functionality comprises increased stability of the mutant casein protein in a liquid.

21. 21 The mutant casein protein in any one of claims 1-18, wherein the mammalian glycan-related protein functionality comprises enhanced casein micelle formation in a liquid.

22. The mutant casein protein in any one of claims 1-18, wherein the mammalian glycan-related protein functionality comprises improved milk production.

23. The mutant casein protein in any one of claims 1-18, wherein the mammalian glycan-related protein functionality comprises improved curd formation in a cheese making process.

24. The mutant casein protein in any one of claims 1-18, wherein the mammalian glycan-related protein functionality comprises an improved dairy characteristic.

25. A casein micelle, comprising:
the mutant κ-casein in any one of claims 1-24; and at least one of αS1-casein, αS2-casein, or β-casein; wherein the mammalian glycan-related protein functionality comprises increased solubility or stability of the casein micelle in a liquid.

26. The casein micelle in claim 25, wherein the increased solubility or stability is evidenced by less than 10% change in the liquid's optical density for at least a week.

27. A vector comprising a nucleic acid construct comprising a polynucleotide sequence encoding the mutant casein protein in claims 1-24.

28. The vector in claim 26, wherein the vector is a recombinant retroviral vector or a TDNA vector.

29. A food composition comprising the mutant κ-casein in claims 1-24.

30. The food composition in claim 29, wherein the food composition further comprises at least one of αS1-casein, αS2-casein, or ß-casein.

31. The food composition in claim 29, wherein the food composition comprises at least one of milk, cheese, ice cream, yogurt, cream, butter, protein powder, protein bar, and baby formula.

32. A method of making a food product, comprising expressing the mutant κ-casein in at least one of claims 1-24 in a plant; isolating the mutant κ-casein from the plant; and mixing the κ-casein with at least one of αS1-casein, αS2-casein, or ß-casein in a solution.

33. A method of making a food product, comprising expressing the mutant κ-casein in at least one of claims 1-24 in a genetically modified microorganism; isolating the mutant κ-casein from the genetically modified microorganism; and mixing the κ-casein with at least one of αS1-casein, αS2-casein, or ß-casein in a solution.

34. The composition of claim 33, wherein microorganism is at least one of yeast, fungi, or bacteria.

35. The method in any one of claims 32-34, wherein the food product comprises at least one of milk, cheese, ice cream, yogurt, cream, butter, protein powder, protein bar, and baby formula.

36. A modified casein protein comprising a modification near the C-terminus of the casein protein to increase hydrophilicity of the C-terminus region.

37. The modified casein protein in claim 36, wherein the modification comprises adding a peptide sequence to the C-terminus of the casein protein.

38. The modified casein protein in claim 37, wherein the peptide sequence has at least two hydrophilic amino acids.

39. The modified casein protein in claim 37, wherein the peptide sequence has at least three hydrophilic amino acids.

40. The modified casein protein in claim 37, wherein the peptide sequence has at least four hydrophilic amino acids.

41. The modified casein protein in claim 37, wherein the peptide sequence has at least five hydrophilic amino acids.

42. The modified casein protein in claim 37, wherein the peptide sequence has at least six hydrophilic amino acids.

43. The modified casein protein in claim 37, wherein the peptide sequence has at least eight hydrophilic amino acids.

44. The modified casein protein in claim 37, wherein the peptide sequence has at least ten hydrophilic amino acids.

45. The modified casein protein in claim 37, wherein the peptide sequence has at least twelve hydrophilic amino acids.

46. The modified casein protein in any one of claims 36-45, wherein the hydrophilic amino acid is at least one of R, H, K, D, E, S, T, N or Q.

47. The modified casein protein in any one of claims 36-46, wherein the peptide sequence is at least 80% identical to SEQ ID No. 8.

48. The modified casein protein in claim 36, wherein the modification comprises mutating at least one hydrophobic amino acid to a hydrophilic amino acid near the C-terminus of the casein protein.

49. The modified casein protein in claim 36, wherein the modification comprises mutating at least two hydrophobic amino acids to hydrophilic amino acids near the C-terminus of the casein protein.

50. The modified casein protein in claim 36, wherein the modification comprises mutating at least three hydrophobic amino acids to hydrophilic amino acids near the C-terminus of the casein protein.

51. The modified casein protein in claim 36, wherein the modification comprises mutating at least four hydrophobic amino acids to hydrophilic amino acids near the C-terminus of the casein protein.

52. The modified casein protein in claim 36, wherein the modification comprises mutating at least five hydrophobic amino acids to hydrophilic amino acids near the C-terminus of the casein protein.

53. The modified casein protein in claim 36, wherein the modification comprises mutating at least six hydrophobic amino acids to hydrophilic amino acids near the C-terminus of the casein protein.

54. The modified casein protein in claim 36, wherein the modification comprises mutating at least eight hydrophobic amino acids to hydrophilic amino acids near the C-terminus of the casein protein.

55. The modified casein protein in claim 36, wherein the modification comprises mutating at least ten hydrophobic amino acids to hydrophilic amino acids near the C-terminus of the casein protein.

56. The modified casein protein in claim 36, wherein the modification comprises mutating at least twelve hydrophobic amino acids to hydrophilic amino acids near the C-terminus of the casein protein.

57. The modified casein protein in any one of claim 48-56, wherein the hydrophobic amino acid is at least one of A, V, I, L, M, F, Y, or W, and the hydrophilic amino acid is at least one of R, H, K, D, E, S, T, N, or Q.

58. The modified casein protein in claim 36, wherein the modification comprises removing at least one hydrophobic amino acid near the C-terminus of the casein protein.

59. The modified casein protein in claim 36, wherein the modification comprises removing at least two hydrophobic amino acids near the C-terminus of the casein protein.

60. The modified casein protein in claim 36, wherein the modification comprises removing at least three hydrophobic amino acids near the C-terminus of the casein protein.

61. The modified casein protein in claim 36, wherein the modification comprises removing at least four hydrophobic amino acids near the C-terminus of the casein protein.

62. The modified casein protein in claim 36, wherein the modification comprises removing at least five hydrophobic amino acids near the C-terminus of the casein protein.

63. The modified casein protein in claim 36, wherein the modification comprises removing at least six hydrophobic amino acids near the C-terminus of the casein protein.

64. The modified casein protein in claim 36, wherein the modification comprises removing at least eight hydrophobic amino acids near the C-terminus of the casein protein.

65. The modified casein protein in claim 36, wherein the modification comprises removing at least ten hydrophobic amino acids near the C-terminus of the casein protein.

66. The modified casein protein in claim 36, wherein the modification comprises removing at least twelve hydrophobic amino acids near the C-terminus of the casein protein.

67. The modified casein protein in any one of claim 58-66, wherein the hydrophobic amino acid is at least one of A, V, I, L, M, F, Y, or W.

68. The modified casein protein in any one of claims 36-67, wherein the modified casein protein is a kappa casein.

69. The modified casein protein in any one of claims 36-68, wherein the modified casein protein is a bovine casein.

70. The modified casein protein in any one of claims 36-68, wherein the modified casein protein is a ruminant casein.

71. The modified casein protein in any one of claims 36-68, wherein the modified casein protein is a human casein.

72. The modified casein protein in any one of claims 36-71, wherein the casein protein is non-glycosylated, under-glycosylated, or differentially-glycosylated.

73. The modified casein protein in any one of claims 36-71, wherein the modification confers mammalian glycan-related protein functionality.

74. The modified casein protein in claim 73, wherein the mammalian glycan-related protein functionality comprises an improved dairy characteristic.

75. A casein micelle, comprising the modified casein in any one of claims 36-74.

76. The casein micelle in claim 75, wherein the modified casein is a κ-casein.

77. The casein micelle in claim 76, further comprising at least one of at least one of αS1-casein, αS2-casein, or ß-casein in a solution.

78. The casein micelle in any one of claims 75-77, wherein the modified κ-casein enhances functionality of the casein micelle.

79. The casein micelle in claim 78, wherein the enhanced functionality comprises increased stability of the casein micelle, solubility of the casein micelle, or improved curd formation in a cheese making process using the casein micelle.

80. A food composition comprising the modified casein in any one of claims 36-74.

81. A food composition comprising the casein micelle in any one of claims 75-79.

82. The food composition in claim 81, wherein the food composition comprises at least one of milk, cheese, ice cream, yogurt, cream, butter, protein powder, protein bar, and baby formula.

83. A method of making a casein micelle in vitro, comprising expressing a modified casein protein in any one of claims 36-74 in a plant, wherein the modified casein protein is κ-casein; isolating the modified κ-casein from the plant; and mixing the modified κ-casein with at least one of at least one of αS1-casein, αS2-casein, or ß-casein in a solution, thereby forming the casein micelle.

84. A method of making a casein micelle in vitro, comprising expressing a modified casein protein in any one of claims 36-74 in a microorganism; wherein the modified casein protein is κ-casein; isolating the modified κ-casein from the microorganism; and mixing the modified κ-casein with at least one of αS1-casein, αS2-casein, or ß-casein in a solution, thereby forming the casein micelle.

85. A method of making a casein micelle in vivo, comprising co-expressing in a plant or microorganism, 1) a modified casein protein in any one of claims 36-74, wherein the modified casein protein is κ-casein, and 2) at least one of αS1-casein, αS2-casein, or ß-casein in a solution, wherein the modified κ-casein and at least one of αS1-casein, αS2-casein, or ß-casein form the casein micelle in vivo.

86. The method in claim 85, wherein the plant is soybean.

87. The method in claim 85, wherein the microorganism is a fungus.

88. The method in claim 85, wherein the microorganism is bacteria.

89. The method in claim 85, wherein the casein is bovine casein.

90. The method in claim 85, wherein the casein is human casein.
